(19) United States
(12) Patent Application Publication
(10) Pub. No.: US 2010/0297051 A1
(43) Pub. Date: Nov. 25, 2010

(54) COSMETIC COMPOSITION COMPRISING AT LEAST ONE ELASTOMERIC ANIONIC POLYURETHANE AND AT LEAST ONE NON-IONIC THICKENER

(76) Inventor: Laetitia FEUILLETTE, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/470,849

(22) Filed: May 22, 2009

(51) Int. Cl. A61K 8/18; A61K 31/74; A61Q 15/00

(52) U.S. Cl. 424/65; 424/78.08

(57) ABSTRACT

The present disclosure relates to a cosmetic composition comprising, in a cosmetically acceptable medium, at least one elastomeric anionic film-forming polyurethane and at least one non-ionic thickening polymer, as well as a hairdressing method for the shaping of the hair comprising the application of this composition.

### FIELD OF THE INVENTION

The present disclosure relates to a cosmetic composition comprising, in a cosmetically acceptable medium, at least one elastomeric film-forming polymer. It also relates to the use of this composition for the formulation of products for shaping, conditioning or care of the hair, for example, for the formulation of hair-styling products.

The present disclosure also relates to a method of conditioning, shaping or caring of the hair using this composition.

Fixing of the hairstyle is an important element of styling, and involves the maintenance of shape. According to the present disclosure, the term “styling composition” refers to any type of hair-dressing composition that can be used for effecting styling.

### TECHNOLOGICAL BACKGROUND

Among the hair care products for hair fixing that are most widely available on the cosmetics market, mention may be made of spray compositions in aerosols or pump-action bottles such as lacquers, sprays or mousses, essentially constituted of a solution that is generally alcoholic or aqueous-alcoholic and of a film-forming polymer that is water-soluble or alcohol-soluble, mixed with various cosmetic additives, or alternatively mention may be made of products that are to be applied by hand, such as gels and waxes.

However, these hair styling formulations, such as the aerosol sprays and lacquers may not always give the hairstyle satisfactory resistance to the various natural everyday movements such as walking, movements of the head or gusts of wind. Moreover, these compositions may give the hair a sensation of stiffness, the so-called “helmet effect”. Styling products also include the styling shampoos, which offer the advantage of combining washing of the hair while permitting shaping of the hair during drying, saving time for the increasingly busy user.

The polymers used conventionally for the formulation of hair styling products are cationic, anionic, amphoteric or non-ionic film-forming polymers, which may lead to the formation of films that are hard and brittle to a varying degree.

When the polymer is too brittle, the percentage elongation at break measured on the film is low, i.e. generally less than 2%, and the hairstyle is not long-lasting.

To overcome this problem, these polymers have already been mixed with plasticizers, obtaining films that are more flexible and are not brittle. These films are deformable and plastic, and after deformation, they only regain their initial form to a slight extent. Although the lasting properties of the hairstyle are improved, they still may not be satisfactory, since the form of the hairstyle changes, under the action of the stresses.

Therefore cosmetic compositions are being sought for the care and/or fixing of the hairstyle which give the hair, in addition to long-lasting fixing, good cosmetic properties, notably conditioning of the hair conferring good disentangling, softness and a pleasant, non-sticky appearance, as well as ease of use and low risk in use.

One approach for achieving this objective is polyurethane technology. The polyurethanes have a characteristic of being very elastic and therefore of forming a stretchable film, which can withstand mechanical stresses. On the hair, this can translate into the formation of a homogeneous, non-brittle sheathing along the fibre.

The problems associated with this polyurethane technology are that the polyurethanes are very hydrophobic and therefore are not dispersible as such in water, or they are often in the form of opaque white latices. In this case, when included in any cosmetic formula, on application there may be formation of a white deposit on the hair, which is unacceptable for the consumer. In contrast to the conventional polyurethanes, the polyurethanes according to the present disclosure are dispersible in water, giving clear or translucent solutions.

However, certain polyurethanes are non-thickening. In this case they may be combined with thickening polymers in the formulations so that they can be manipulated easily and deposited on the hair without running.

The use of water-soluble or water-dispersible polymers as thickening agent for aqueous media, and optionally crosslinked polymers such as polycarboxyvinylic acids and CARBOPOL® Polymers, said polymers having a considerable chain length and a high molecular weight, is known.

Now, in certain cases, the anionic thickeners are incompatible with the polyurethanes, and may lead to compositions that are unsatisfactory for the consumer.

It was discovered, unexpectedly, that by combining particular anionic polyurethanes with non-ionic thickeners it can be possible to obtain thickened compositions, such as gels, that are translucent, or even transparent, having sufficient viscosity and an appearance that is satisfactory for the consumer, while solving at least one of the drawbacks listed above. When applied to the hair, these thickened compositions lead to flexible, long-lasting fixing.

### DESCRIPTION OF THE INVENTION

The present disclosure relates to a cosmetic composition comprising, in a cosmetically acceptable medium, at least one elastomeric anionic film-forming polyurethane (A) and at least one non-ionic thickening polymer, wherein the at least one elastomeric anionic film-forming polyurethane (A) comprises: (i) at least one side chain comprising at least one ethylene oxide unit, wherein the at least one side chain
represents from 12 to 80% by weight of polyurethane (A), and
has a molecular weight ranging from 1000 g/mol to 30,000 g/mol; and

(ii) one main chain comprising units derived from:
a polypropylene glycol (PPG) and optionally a second non-ionic polyol,
a dihydroxy-carboxylic acid, and
at least one di-isocyanate; and optionally
a chain extender organic polyamine comprising an average of at least two primary amine groups.

This composition may be used to obtain a flexible, non-brittle film on human keratinous fibers, which may allow the human keratinous fibers to move perfectly.

In addition, the composition according to the present disclosure may be used for conditioning the human keratinous fibers.

Another aspect of the present disclosure relates to a method of shaping or maintaining the hairstyle comprising applying the disclosed composition to human keratinous fibers.

### DETAILED DESCRIPTION OF THE EMBODIMENTS OF THE INVENTION

### The Chemistry of Film-Forming Elastomeric Polyurethane

The at least one elastomeric anionic film-forming polyurethane (A) may be prepared according to the method described in International Patent Application Publication Nos: WO2006/124250 and WO03/087183.

In at least one embodiment, the at least one elastomeric anionic film-forming polyurethane (A) comprises

(i) at least one side chain comprising at least one ethylene oxide unit, wherein the at least one side chain
represents from 12 to 80% by weight of polyurethane (A), and
has a molecular weight ranging from 1000 g/mol to 30,000 g/mol; and

(ii) one main chain comprising units derived from:
a polypropylene glycol (PPG) and optionally a second non-ionic polyol,
a dihydroxy-carboxylic acid such as a dimethylolpropionic acid (DMPA),
at least one di-isocyanate, for instance, a isophorone diisocyanate; and
a chain extender organic polyamine having an average of at least 2 primary amine groups.

The at least one elastomeric anionic film-forming polyurethane (A) is soluble or dispersible, either in an aqueous medium (100% water), or in a water/ethanol solvent mixture containing at most 30 wt. % of ethanol. For example, at least 10 g of polymer (A) is soluble or dispersible in 90 grams of water or of water/ethanol mixture.

The at least one elastomeric anionic film-forming polyurethane (A) can be partially or totally neutralized or over-neutralized with inorganic or organic bases, for instance, with soda and/or triethylamine, in an amount making it possible to obtain, for example, a degree of neutralization of the anionic functions of the polymer ranging from 0 to 150%, such as ranging from 50 to 100%. The “degree of neutralization to 150%” is obtained with an amount of inorganic or organic base equal to 1.5 times the amount of base required to obtain the degree of neutralization to 100%.

The result of this is that the at least one elastomeric anionic film-forming polyurethane (A) is a salt composed of two parts: one anionic polyurethane and a cationic ion, metallic or organic.

### Mechanical Profile of the Film-Forming Elastomeric Polyurethane

In at least one embodiment, in the compositions according to the present disclosure, the film obtained by drying the at least one elastomeric anionic film-forming polyurethane (A) at room temperature (24° C.±2° C.) and at a relative humidity of 48%±5%, has a mechanical profile comprising:

(a) an elongation at break (ε_{b}) greater than or equal to 730%±5% (e.g. −5% of 730% represents 694%).

(b) an instantaneous recovery (Rᵢ) greater than or equal to 70%±5%, after elongation of 150%; and

(c) a recovery (R₃₀₀) at 300 seconds greater than 80%±5%.

### Measurement of the Parameters

For the purpose of the present disclosure, the expression, “film obtained by drying at room temperature (24° C.±2° C.) and at a relative humidity of 48%±5%” means the film obtained, in these conditions, from a mixture comprising 6% of active substance (a.s.) of the elastomeric anionic film-forming polyurethane (A), in a solvent (S). The amount of active substance is understood as being relative to the total weight of solvent (S) and the amount of mixture is suitable for obtaining, in a Teflon matrix, a film with thickness of 500 μm±50 μm, after drying the mixture, said drying being continued until there is no longer any change in weight of the film, which represents approximately 10 days.

The solvent (S) is a mixture ethanol/water comprising less than 30% by weight of water.

For the purpose of the present disclosure, the expression “elongation at break” and the “degree of recovery” are evaluated via the tests described below.

To measure the instantaneous recovery and the recovery at 300 seconds, the polymer film obtained is cut into test pieces of rectangular shape, 80 mm long and 15 mm wide.

To measure the elongation, the polymer film obtained is cut into dumbbell-shaped test pieces.

The tests are performed on apparatus marketed under the name LLOYD or marketed under the name ZWICK in the same conditions of temperature and of humidity as for drying, i.e. a temperature of 24° C.±2° C. and a relative humidity of 48%±5%.

The test pieces are stretched at a speed of 20 mm/min and the distance between the jaws is 50 mm±1 mm.

The elongation at break represents the ratio of the maximum elongation of the film before it breaks to its initial length measured before undergoing deformation.

The procedure for determining the instantaneous recovery (Rᵢ) is as follows:
stretch the test piece 150% (εₘₐₓ) i.e. 1.5 times its initial length (I₀),
release the stress, imposing a return speed equal to the pulling speed, i.e. 20 mm/min, and measure the percentage elongation of the test piece, after returning to zero load (εᵢ).

The % instantaneous recovery (Rᵢ) is given by the following formula:

Rᵢ=((εₘₐₓ−εᵢ)/εₘₐₓ)×100

To determine the recovery at 300 seconds, it is held at zero stress for an additional 300 seconds, the test piece having undergone the preceding operations, and its percentage elongation is measured (ε₃₀₀ₛ).

The recovery at 300 seconds as a percentage (R₃₀₀ₛ) is given by the following formula:

R₃₀₀ₛ=((εₘₐₓ−ε₃₀₀ₛ)/εₘₐₓ)×100

In the compositions according to the present disclosure, the at least one elastomeric anionic film-forming polyurethane (A) may be present in a total amount ranging from 0.05 to 20 wt. %, for instance, ranging from 0.1 to 15 wt. % or ranging from 1 to 10 wt. % relative to the total weight of the composition.

### Non-Ionic Thickening Polymer

According to the present disclosure, any type of non-ionic thickening polymer, whether associative or non-associative, can be used.

In one embodiment, the at least one non-ionic thickening polymer is chosen from associative thickening polymers. They may include at least one hydrophilic sequence and at least one hydrophobic sequence.

The at least one hydrophobic sequence may be chosen from alkyl, alkenyl, alkaryl or aralkyl chains having from 8 to 30 carbon atoms.

The at least one hydrophilic sequence may be chosen from polyethylene oxide, a polysaccharide, a polyamide such as polyacrylamide, a polyester and mixtures thereof, for example, a polyethylene oxide having from 2 to 500 mol of ethylene oxides.

The bond or bonds between hydrophilic sequence and hydrophobic sequence may be chosen from, without imposing any limitation, ester, ether, urea, amide, and urethane type and mixtures thereof. 1) Non-Associative Thickening Polymer

For the purpose of the present disclosure, the expression “non-associative polymer” means a polymer not containing simultaneously at least one C₈-C₃₀ fatty chain and at least one hydrophilic unit.

The non-associative non-ionic polymers according to the present disclosure can be of natural or synthetic origin.

They may be selected from:
non-ionic homopolymers and copolymers containing monomers with an ethylenic unsaturation of the ester and/or amide type,
homo- or copolymers of vinyl pyrrolidone,
polysaccharides.

Among the non-ionic homopolymers or copolymers containing monomers with an ethylenic unsaturation of the ester and/or amide type, exemplary mention may be made of the polyamides, such as the products sold under the names: CYANAMER P250 by the company CYTEC (polyacrylamide); methyl methacrylate/ethylene glycol dimethacrylate copolymers (PMMA MBX-8C by the company US COSMETICS); butyl methacrylate/methyl methacrylate copolymers (ACRYLOID B66 by the company ROHM & HAAS), poly(methyl methacrylate) (BPA 500 by the company KOBO).

The homo- or copolymers of vinyl pyrrolidone may be selected from the crosslinked vinyl pyrrolidone homopolymers such as POLYMER ACP-10 marketed by ISP.

The polysaccharides may be selected from glucans, modified or unmodified starches (such as those obtained, for example, from cereals such as wheat, maize or rice, from legumes such as blond pea, from tubers such as potatoes or manioc), amylose, amylopectin, glycogen, dextrans, celluloses and their derivatives (methylcelluloses, hydroxyalkylcelluloses, ethylhydroxyethyl celluloses), mannans, xylans, lignins, arabans, galactans, galacturonans, chitin, chitosans, glucuronoxylans, arabinoxylans, xyloglucans, glucomannans, pectic acids and pectins, alginic acid and alginates, arabinogalactans, glycosaminoglucans, carrageenins, agars, gum arabic, gum tragacanth, carrageenan gums, Ghatti gums, karaya gums, carob gums, galactomannans such as guar gums and their non-ionic derivatives (hydroxypropyl guar), and scleroglucan or xanthan gums, and mixtures thereof.

The compounds of this type that can be used in the present disclosure may be selected from those described in “Encyclopedia of Chemical Technology”, Kirk-Othmer, Third Edition, 1982, volume 3, pp. 896-900, and volume 15, pp 439-458, in “Polymers in Nature”, by E. A. MacGregor and C. T. Greenwood, published by John Wiley & Sons, Chapter 6, pp 240-328, 1980 and in “Industrial Gums—Polysaccharides and their Derivatives”, Edited by Roy L. Whistler, Second Edition, publ. Academic Press Inc..

Starches, guar gums, celluloses and their derivatives may be used.

The polysaccharides can be modified or unmodified.

Unmodified guar gums are for example the products sold under the name VIDOGUM GH 175 by the company UNIPECTINE, and under the names MEYPRO-GUAR 50 and JAGUAR C by the company RHODIA CHIMIE.

The modified non-ionic guar gums may be modified with C₁-C₆ hydroxyalkyl groups.

Among the hydroxyalkyl groups, we may mention for example the hydroxymethyl, hydroxyethyl, hydroxypropyl and hydroxybutyl groups.

These guar gums are well known to one of ordinary skill in the art and can, for example, be prepared by reacting the corresponding oxides of alkenes, for example propylene oxides, with guar gum so as to obtain a guar gum modified with hydroxypropyl groups.

The degree of hydroxyalkylation, which corresponds to the number of molecules of alkylene oxide consumed by the number of free hydroxyl functions present on the guar gum, may range from 0.4 to 1.2.

These non-ionic guar gums, optionally modified with hydroxyalkyl groups, are for example sold under the trade names JAGUAR HP8, JAGUAR HP60 and JAGUAR HP1 20, JAGUAR DC 293 and JAGUAR HP 105 by the company RHODIA CHIMIE or under the name GALACTASOL 4H4FD2 by the company AQUALON.

Among the celluloses, non-limiting mention may be made of the hydroxyethyl celluloses and the hydroxypropyl celluloses. For example, the products sold under the names KLUCEL EF, KLUCEL H, KLUCEL LHF, KLUCEL MF, KLUCEL G, by the company AQUALON; CELLOSIZE POLYMER PCG-10 by the company AMERCHOL.

2) Associative Thickening Polymer

The associative polymers may be selected from:

(1) the celluloses modified with groups comprising at least one fatty chain, among which non-limiting mention may be made of:
the hydroxyethyl celluloses modified with groups having at least one fatty chain such as alkyl, aralkyl, alkaryl groups, or mixtures thereof, wherein the alkyl groups may be chosen from C₈-C₂₂ alkyl groups, such as the product NATROSOL PLUS GRADE 330 CS (C₁₆ alkyls) sold by the company AQUALON, or the product BERMOCOLL EHM 100 sold by the company BEROL NOBEL,
those modified with alkyl phenol polyalkylene glycol ether groups, such as the product AMERCELL POLYMER HM-1500.

(2) the non-ionic guar gums comprising at least one fatty chain such as:
the hydroxypropyl guars modified with groups having at least one fatty chain such as the product ESAFLOR HM 22 (C₂₂ alkyl chain) sold by the company LAMBERTI, the products RE210-18 (C₁₄ alkyl chain) and RE205-1 (C₂₀ alkyl chain) sold by the company RHONE POULENC.

(3) copolymers of vinyl pyrrolidone and hydrophobic fatty-chain monomers, among which non-limiting mention may be made of, as examples:
the products ANTARON V216 or GANEX V216 (vinyl pyrrolidone/hexadecene copolymer) sold by the company I.S.P.
the products ANTARON V220 or GANEX V220 (vinyl pyrrolidone/eicosene copolymer) sold by the company I.S.P.

(4) copolymers of methacrylates or acrylates of C₁-C₆ alkyls and amphiphilic monomers comprising at least one fatty chain, for example, the oxyethylenated methyl acrylate/stearyl acrylate copolymer sold by the company GOLDSCHMIDT under the name ANTIL.

(5) copolymers of hydrophilic methacrylates or acrylates and hydrophobic monomers comprising at least one fatty chain, for example the polyethylene glycol methacrylate/lauryl methacrylate copolymer.

(6) polyether polyurethanes comprising, in their chain, simultaneously, hydrophilic sequences such as polyoxyethylenated nature and hydrophobic sequences, which may be chosen from aliphatic sequences, cycloaliphatic sequences, and aromatic sequences.

(7) polymers with an aminoplast ether backbone comprising at least one fatty chain, such as the compounds PURE THIX offered by the company SOD-CHEMIE.

The polyether polyurethanes have at least two lipophilic hydrocarbon chains, having from 6 to 30 carbon atoms, separated by a hydrophilic sequence, and the hydrocarbon chains can be pendant chains or chains at the end of a hydrophilic sequence. For instance, it is possible for at least one pendant chains to be provided. Moreover, the polymer can have a hydrocarbon chain at one end or at both ends of a hydrophilic sequence.

The polyether polyurethanes can be multiblock copolymers such as in the form of triblock copolymers. The hydrophobic sequences can be at each end of the chain (for example: triblock copolymer with central hydrophilic sequence) or distributed both at the ends and in the chain (multiblock copolymer, for example). These same polymers can also be as grafts or star-shaped.

The non-ionic fatty-chain polyether polyurethanes may be triblock copolymers wherein the hydrophilic sequence is chosen from polyoxyethylenated chains having from 50 to 1000 oxyethylenated groups. The nonionic polyether polyurethanes have a urethane linkage between the hydrophilic sequences, hence the name.

By extension, the non-ionic fatty-chain polyether polyurethanes include those in which the hydrophilic sequences are bound to the lipophilic sequences by other chemical bonds.

Non-limiting examples of non-ionic fatty-chain polyether polyurethanes that can be used in the present disclosure include: RHEOLATE.205 with urea function sold by the company RHEOX or alternatively RHEOLATES.208, 204 or 212, as well as ACRYSOL RM. We may also mention the product ELFACOS T210 with C₁₂-C₁₄ alkyl chain and the product ELFACOS T212 with C₁₈ alkyl chain from AKZO.

The product DW 1206B from RÖHM & HAAS with C₂₀ alkyl chain and with a urethane linkage, offered at 20% of dry matter in water, can also be used.

Solutions or dispersions of these polymers in, for instance, water or aqueous-alcoholic medium may also be used. Non-limiting mention may be made of RHEOLATE, RIHEOLATE.278 and RHEOLATE.244 sold by the company RHEOX. The product DW 1206F and the product DW 1206J offered by the company RÖHM & HAAS can also be used.

The polyether polyurethanes that can be used according to the present disclosure may be those described in the article of G. Fonnum, J. Bakke and Fk. Hansen—Colloid Polym. Sci 271, 380-389 (1993).

As a non-limiting example, according to the present disclosure, a polyether polyurethane that is prepared from polycondensation of at least three compounds comprising (i) at least one polyethylene glycol containing from 150 to 180 mol of ethylene oxide, (ii) stearyl alcohol or decyl alcohol and (iii) at least one diisocyanate.

Non-limiting mention may be made of polyether polyurethanes sold by the company RÖHM & HAAS under the names ACULYN 46 and ACULYN 44. ACULYN 46 is a polycondensate of polyethylene glycol with 150 or 180 mol of ethylene oxide, of stearyl alcohol and of methylene bis(4-cyclohexyl-isocyanate) (SMDI), at 15 wt. % in a matrix of maltodextrin (4%) and water (81%); ACULYN 44 is a polycondensate of polyethylene glycol with 150 or 180 mol of ethylene oxide, of decyl alcohol and of methylene bis(4-cyclohexylisocyanate) (SMDI), at 35 wt. % in a mixture of propylene glycol (39%) and water (26%).

In at least one embodiment, the at least one associative polymer is chosen from:
Non-ionic polyether polyurethanes having at least one polyoxyethylenated chain and at least one fatty chain such as C8-C30 alkyl or alkenyl groups, such as the products ACULYN 44 and ACULYN 46 sold by the company RÖHM & HAAS
The polyurethane polyether with alpha-omega stearyl polyoxyethylenated termination RHEOLATE FX 1100, marketed by the company ELEMENTIS
As another non-limiting example, the polymer DERMOTHIX 100 sold by the company ALZO, the molecule of which has two oxyethylenated units each joined to a C18 hydrocarbon group at the chain end and joined together via a polyurethane sequence.

In another embodiment, the non-ionic thickening polymer is selected from:

(1) celluloses modified with groups comprising at least one fatty chain,

(2) polyether polyurethanes having both hydrophilic sequences such as a polyoxyethylenated nature and hydrophobic sequences in their chain.

The non-ionic thickening polymer or polymers is/are present in an amount ranging from 0.01 to 5 wt. %, for instance, in an amount ranging from 0.1 to 2 wt. %, relative to the total weight of the composition.

### Cosmetic Additive

The cosmetically acceptable medium may be chosen from water and/or at least one organic solvent, such as selected from the C₂-C₄ alcohols, for instance, ethanol, the polyols and ethers of polyols such as propylene glycols, polyethylene glycols, glycerol, acetone, propylene carbonate and benzyl alcohol.

The compositions according to the present disclosure can also contain other cosmetically acceptable additives, for example ionic or non-ionic surfactants; cationic, anionic or amphoteric thickeners; penetrating agents; perfumes; colorants; plasticizers; buffers; and various usual additives such as waxes; volatile or non-volatile, cyclic or linear or branched silicones, which may be organically modified such as alkoxylated or modified with amine groups or unmodified; ceramides, pseudoceramides; vegetable, mineral or synthetic oils; vitamins or provitamins such as panthenol; opacifiers; reducing agents; emulsifiers; preservatives; mineral fillers; nacres; glitter; sun filters; proteins; anionic, non-ionic, cationic or amphoteric fixing polymers different from anionic polyurelhanes (A); hydrating agents; emollients; softening agents; antifoaming agents; antiperspirants; anti-free-radical agents; bactericides; sequestering agents; anti-dandruff agents; antioxidants; alkalizing agents; acidifying agents; and any other additive conventionally used in cosmetic compositions intended to be applied on the hair.

The surfactants for use in the composition according to the present disclosure can be anionic, non-ionic, amphoteric or cationic surfactants, or mixtures thereof.

Among the anionic surfactants that can be used, individually or mixed, within the scope of the present disclosure, non-limiting mention may be made of salts, for example, the salts of alkali metals such as sodium salts, ammonium salts, salts of amines, salts of amino-alcohols, or salts of alkaline-earth metals, for example of magnesium, of the following compounds: alkyl sulphates, alkyl ether sulphates, alkylamidoether sulphates, alkarylpolyether sulphates, monoglyceride-sulphates; alkyl sulphonates, alkylamide sulphonates, alkaryl sulphonates, α-olefin-sulphonates, paraffin-sulphonates; alkyl sulphosuccinates, alkyl ether sulphosuccinates, alkylamide-sulphosuccinates; alkyl sulphoacetates; acyl sarconisates; and the acylglutamates, the alkyl and acyl groups of all these compounds having from 6 to 24 carbon atoms and the aryl group such as a phenyl or benzyl group.

In addition, within the scope of the present disclosure, C₆-C₂₄ alkyl esters of polyglycoside-carboxylic acids such as alkyl glucoside-citrates, alkyl polyglycoside-tartrates, and alkyl polyglycoside-sulphosuccinates; alkylsulphosuccinamates, acylisethionates and N-acyltaurates, the alkyl or acyl group of all these compounds having from 12 to 20 carbon atoms. Among other anionic surfactants that can be used, non-limiting mention may be made of the acyl lactylates in which the acyl group has from 8 to 20 carbon atoms.

Moreover, we may also mention the alkyl-D-galactoside uronic acids and their salts as well as the polyoxyalkylenated alkyl(C₆-C₂₄)ether-carboxylic acids, the polyoxyalkylenated alkyl(C₆-C₂₄)aryl(C₆-C₂₄)ether-carboxylic acids, the polyoxyalkylenated alkyl(C₆-C₂₄)amidoether carboxylic acids and their salts, such as those having from 2 to 50 ethylene oxide groups, and mixtures thereof.

The non-ionic surfactants that can be used within the scope of the present disclosure are also compounds that are well known per se (in this connection, see for instance, “Handbook of Surfactants” by M. R. PORTER, publ. Blackie & Son (Glasgow and London), 1991, pp 116-178). They may be selected from the alcohols, the alpha-diols, the alkyl(C₁-C₂₀) phenols or the polyethoxylated, polypropoxylated or polyglycerolated fatty acids, having a fatty chain with for example from 8 to 18 carbon atoms, with the number of ethylene oxide groups or propylene oxide groups ranging from 2 to 50 and the number of glycerol groups ranging from 2 to 30. We may also mention the copolymers of ethylene oxide and propylene oxide, the condensates of ethylene oxide and propylene oxide on fatty alcohols; the polyethoxylated fatty amides having from 2 to 30 mol of ethylene oxide; the polyglycerolated fatty amides having on average 1 to 5 glycerol groups such as 1.5 to 4 glycerol groups; the polyethoxylated fatty amines having 2 to 30 mol of ethylene oxide; the ethoxylated sorbitan fatty acid esters having from 2 to 30 mol of ethylene oxide; the sucrose fatty acid esters, the polyethylene glycol fatty acid esters, the alkyl(C₆-C₂₄)polyglucosides, the derivatives of N-alkyl(C₆-C₂₄)glucamine, the oxides of amines such as the oxides of alkyl(C₁₀-C₁₄)amines or the oxides of N-acyl(C₁₀-C₁₄)aminopropylmorpholine; and mixtures thereof.

The amphoteric surfactants, suitable for the present disclosure, may be chosen from derivatives of secondary or tertiary aliphatic amines, wherein the aliphatic group is a linear or branched chain comprising 8 to 22 carbon atoms and containing at least one water-solubilizing anionic group such as, for example, a carboxylate, sulphonate, sulphate, phosphate or phosphonate group; we may also mention the alkyl(C₈-C₂₀)betaines, the sulphobetaines, the alkyl(C₈-C₂₀)amidoalkyl(C₆-C₈)-betaines or the alkyl(C₈-C₂₀)amidoalkyl(C₆-C₈)sulphobetaines; and mixtures thereof.

Among the derivatives of amines, non-limiting mention may be made of the products marketed under the name MIRANOL®, as described in U.S. Pat. No. 2,528,378 and U.S. Pat. No. 2,781,354 and classified in the CTFA Dictionary, 3rd edition, 1982, under the designations Amphocarboxy-glycinate and Amphocarboxypropionate with the respective structures (1) and (2):

R₂—CONHCH₂CH₂—N⁺(R₃)(R₄)(CH₂COO⁻) (1)

wherein:

R₂ is chosen from the alkyl groups derived from an R₂—COOH acid present in hydrolysed copra oil, heptyl groups, nonyl groups, or undecyl groups;

R₃ is chosen from beta-hydroxyethyl groups; and

R₄ is chosen from carboxymethyl groups; and

R₂—CONHCH₂CH₂—N(B)(C) (2)

wherein:

B is chosen from —CH₂CH₂OX′,

C is chosen from —(CH₂)_{z}—Y′, with z being chosen from 1 and 2,

X′ is chosen from the —CH₂CH₂—COOH groups and a hydrogen atom,

Y′ is chosen from —COOH and —CH₂—CHOH—SO₃H,

R₂ is chosen from the alkyl groups of an R₂—COOH acid present in copra oil or in hydrolysed linseed oil, alkyl groups, such as C₁₇ and its iso form or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

As a non-limiting example, the cocoamphodiacetate marketed under the trade name MIRANOL® “C2M concentré” by the company RHODIA.

Among the amphoteric surfactants, the alkyl(C₈-C₂₀)betaines such as cocobetaine, the alkyl(C₈-C₂₀)amidoalkyl(C₆-C₈)betaines such as cocamidobetaine, the alkylamphodiacetates such as disodium cocoamphodiacetate, and mixtures thereof.

The composition according to the present disclosure can additionally contain at least one cationic surfactant that are well known per se, such as the salts of primary, secondary or tertiary fatty amines, optionally polyoxyalkylenated, the quaternary ammonium salts such as the tetraalkylammonium, alkylamidoalkyltrialkyl-ammonium, trialkylbenzylammonium, trialkylhydroxyalkyl-ammonium or alkylpyridinium chlorides or bromides, the derivatives of imidazoline; or the oxides of amines of cationic character.

The non-ionic, anionic, amphoteric and cationic surfactants described above can be used individually or mixed and their amount is ranging from 0.01 and 30 wt. %, for example, ranging from 0.05 and 20 wt. % such as ranging from 0.1 and 10 wt. % relative to the total weight of the composition.

The silicones for use as additives in the cosmetic compositions of the present disclosure are volatile or non-volatile silicones, cyclic, linear or branched, unmodified or modified with organic groups, having a viscosity ranging from 5.10⁻⁶ to 2.5 m²/s at 25° C., such as ranging from 1.10⁻⁵ to 1 m²/s.

The silicones that can be used according to the present disclosure can be soluble or insoluble in the composition, for instance, it can be polyorganosiloxanes that are insoluble in the composition of the present disclosure. They can be in the form of oils, waxes, resins or gums.

The organopolysiloxanes are defined in more detail in the work of Walter NOLL “Chemistry and Technology of Silicones” (1968), Academic Press. They can be volatile or non-volatile.

When they are volatile, the silicones may be selected from those with a boiling point between 60° C. and 260° C., non-limiting examples include:

(i) the cyclic silicones having from 3 to 7, such as from 4 to 5 silicon atoms. These are, for example, the octamethylcyclotetrasiloxane marketed under the name VOLATILE SILICONE® 7207 by UNION CARBIDE or SILBIONE® 70045 V2 by RHODIA, the decamethylcyclopentasiloxane marketed under the name VOLATILE SILICONE® 7158 by UNION CARBIDE, and SILBIONE® 70045 V5 by RHODIA, and mixtures thereof.

We may also mention the cyclocopolymers of the dimethylsiloxane/methylalkylsiloxane type, such as the SILICONE VOLATILE® FZ 3109 marketed by the company UNION CARBIDE, of formula:

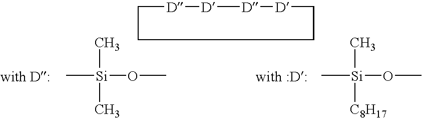

Non-limiting mention may also be made of the mixtures of cyclic silicones with organic compounds derived from silicon, such as the mixture of octamethylcyclotetrasiloxane and tetratrimethylsilylpentaerythritol (50/50) and the mixture of octarnethylcyclotetrasiloxane and oxy-1,1′-(hexa-2,2,2′2′3,3′-trimethylsilyloxy) bis-neopentane.

(ii) the volatile linear silicones having 2 to 9 silicon atoms and with a viscosity less than or equal to 5.10⁻⁶ m²/s at 25° C. These include, for example, the decamethyltetrasiloxane marketed under the name “SH 200” by the company TORAY SILICONE. Silicones included in this class are also described in the article published in Cosmetics and Toiletries, Vol. 91, January 76, p. 27-32—TODD & BYERS “Volatile Silicone fluids for cosmetics”.

Non-volatile silicones may also be used, non-limiting examples include polyalkylsiloxanes, polyarylsiloxanes, polyalkarylsiloxanes, silicone gums and resins, polyorganosiloxanes modified with organofunctional groups and mixtures thereof.

For example, these silicones may be selected from the polyalkylsiloxanes such as the polydimethylsiloxanes with trimethylsilyl end groups. The viscosity of the silicones is measured at 25° C. according to standard ASTM 445 Appendix C.

Among these polyalkylsiloxanes, as non-limiting examples, the following commercial products:
the SILBIONE® oils of series 47 and 70 047 or the MIRASIL® oils marketed by RHODIA, for example the oil 70 047 V 500 000;
the oils of the MIRASIL® series marketed by the company RHODIA;
the oils of the 200 series from the company DOW CORNING such as DC200 with a viscosity of 60 000 mm²/s;
the VISCASIL® oils from GENERAL ELECTRIC and certain oils of the SF series (SF 96, SF 18) from GENERAL ELECTRIC.

We may also mention the polymethylsiloxanes with dimethylsilanol end groups known under the name dimethiconol (CTFA), such as the oils of series 48 from the company RHODIA.

In this class of polyalkylsiloxanes, we may also mention the products marketed under the names “ABIL WAX® 9800 and 9801” by the company GOLDSCHMIDT, which are polyalkyl (C₁-C₂₀) siloxanes.

The polyalkarylsiloxanes are selected, for instance, from the polydimethyl/methylphenylsiloxanes, the polydimethyl/diphenylsiloxanes, linear and/or branched with viscosity ranging from 1.10⁻⁵ to 5.10⁻² m²/s at 25° C.

Among these polyalkarylsiloxanes we may mention as non-limiting examples the products marketed under the following names:
the SILBIONE® oils of the 70 641 series from RHODIA;
the oils of the series RHODORSIL® 70 633 and 763 from RHODIA;
the oil DOW CORNING 556 COSMETIC GRADE FLUID from DOW CORNING;
the silicones of the PK series from BAYER such as the product PK20;
the silicones of the PN, PH series from BAYER such as the products PN1000 and PH1000;
certain oils of the SF series from GENERAL ELECTRIC such as SF 1023, SF 1154, SF 1250, SF 1265.

The silicone gums that can be used according to the present disclosure include polyorganosiloxanes, with high number-average molecular weights between 200,000 and 1,000,000 used alone or mixed in a solvent. Said solvent can be selected from the volatile silicones, the polydimethylsiloxane (PDMS) oils, the polyphenylmethylsiloxane (PPMS) oils, the isoparaffins, the polyisobutylenes, methylene chloride, pentane, dodecane, tridecane or mixtures thereof.

We may mention the following products:
polydimethylsiloxane gums,
polydimethylsiloxane/methylvinylsiloxane gums,
polydimethylsiloxaneldiphenylsiloxane gums,
polydimethylsiloxane/phenylmethylsiloxane gums,
polydimethylsiloxane/diphenylsiloxane/methylvinylsiloxane gums.

Products that can be used according to the present disclosure are mixtures such as:
mixtures formed from a polydimethylsiloxane hydroxylated at the chain end, or dimethiconol (CTFA) and a cyclic polydimethylsiloxane also called cyclomethicone (CTFA) such as the product Q2 1401 marketed by the company DOW CORNING;
mixtures of a polydimethylsiloxane gum and a cyclic silicone such as the product SF 1214 Silicone Fluid from the company GENERAL ELECTRIC; said product is a gum SF 30 corresponding to a dimethicone, having a number-average molecular weight of 500,000 dissolved in oil SF 1202 Silicone Fluid corresponding to decamethylcyclopentasiloxane;
mixtures of two PDMS with different viscosities, for instance, a PDMS gum and a PDMS oil, such as the product SF 1236 from the company GENERAL ELECTRIC. The product SF 1236 is a mixture of a gum SE 30 defined above having a viscosity of 20 m²/s and an oil SF 96 with a viscosity of 5.10⁻⁶ m²/s. This product may have 15% of gum SE 30 and 85% of oil SF 96.

The resins of organopolysiloxanes that can be used according to the present disclosure are crosslinked siloxane systems containing the units:

(R)₂SiO_{2/2}, (R)₃SiO_{1/2}, RSiO_{3/2} and SiO_{4/2}

wherein R is chosen from hydrocarbon groups with 1 to 16 carbon atoms and phenyl groups. Among these products, non-limiting mention may be made of the ones in which R is chosen from C₁-C₄ lower alkyl groups, such as methyl or a phenyl group.

We may mention among these resins the product marketed under the name “DOW CORNING 593” or those marketed under the names “SILICONE FLUID SS 4230 and SS 4267” by the company GENERAL ELECTRIC and which are silicones of dimethyl/trimethyl siloxane structure.

We may also mention the resins of the trimethylsiloxysilicate type marketed under the names X22-4914, X21-5034 and X21-5037 by the company SHIN-ETSU.

The organically modified silicones that can be used according to the present disclosure are silicones as defined previously, having in their structure at least one organofunctional groups fixed via a hydrocarbon group.

Among the organically modified silicones, we may mention polyorganosiloxanes having:
polyethyleneoxy and/or polypropyleneoxy groups optionally with C₆-C₂₄ alkyl groups such as the products called dimethicone copolyol marketed by the company DOW CORNING under the name DC 1248 or the SILWET® oils L 722, L 7500, L 77, L 711 from the company UNION CARBIDE and the alkyl (C₁₂)-methicone copolyol marketed by the company DOW CORNING under the name Q2 5200;
substituted or unsubstituted amine groups such as the products marketed under the name GP 4 Silicone Fluid and GP 7100 by the company GENESEE or the products marketed under the names Q2 8220 and DOW CORNING 929 or 939, or DOW CORNING 2-8299 by the company DOW CORNING or the product marketed under the name BELSIL ADM LOG 1 by the company WACKER. The substituted amine groups may be chosen from C₁-C₄ aminoalkyl groups;
thiol groups, such as the products marketed under the names GP 72A and GP 71 from GENESEE.
alkoxylated groups, such as the product marketed under the name “SILICONE COPOLYMER F-755” by SWS SILICONES and ABIL WAX® 2428, 2434 and 2440 by the company GOLDSCHMIDT;
hydroxylated groups, such as the polyorganosiloxanes with hydroxyalkyl function described in French Patent Application No. 8 516 334;
alkoxyalkyl groups, for example the polyorganosiloxanes described in U.S. Pat. No. 4,957,732;
anionic groups of the carboxyl type, for example as in the products described in patent EP 186 507 of the company CHISSO CORPORATION, or of alkyl-carboxyl type such as those present in the product X-22-3701E from the company SHIN-ETSU; 2-hydroxyalkylsulphonate; 2-hydroxyalkylthiosulphate such as the products marketed by the company GOLDSCHMIDT under the names ABIL® S201 and ABIL® S255;
hydroxyacrylamino groups, such as the polyorganosiloxanes described in application EP 342 834. We may mention, for example, the product Q2-8413 from the company DOW CORNING.

The silicones as described above can be used alone or mixed, in an amount ranging from 0.01 to 20 wt. %, such as ranging from 0.1 to 5 wt. %.

The compositions of the present disclosure can also contain non-silicone fatty substances, such as mineral, vegetable, animal and synthetic oils; waxes, fatty esters, ethoxylated or non-ethoxylated fatty alcohols, and fatty acids.

As oils for use in the composition of the present disclosure, non-limiting mention may be made of:
hydrocarbon oils of animal origin, such as perhydrosqualene;
hydrocarbon oils of vegetable origin, such as the liquid triglycerides of fatty acids having from 4 to 10 carbon atoms such as the triglycerides of heptanoic or octanoic acids or alternatively, for example, sunflower oil, maize oil, soya oil, cucurbit oil, grapeseed oil, sesame oil, hazelnut oil, apricot oil, macadamia oil, arara oil, sunflower oil, castor oil, avocado oil, the triglycerides of caprylic/capric acids such as those sold by the company Stearineries Dubois or those sold under the names MIGLYOL® 810, 812 and 818 by the company Dynamit Nobel, jojoba oil, shea butter oil;
linear or branched hydrocarbons, of mineral or synthetic origin, such as the volatile or non-volatile paraffin oils, and their derivatives, petroleum jelly, the polydecenes, hydrogenated polyisobutene such as PARLEAM®; the isoparaffins such as isohexadecane and isodecane.
the fluorinated oils, partially hydrocarbonized and/or siliconized, such as those described in document JP-A-2-295912; as fluorinated oils, we may also mention perfluoromethylcyclopentane and perfluoro-1,3-dimethylcyclohexane, sold under the names “FLUTEC® PC1” and “FLUTEC® PC3” by the company BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the company 3M, or alternatively bromoperfluorooctyl sold under the name “FORALKYL®” by the company Atochem; nonafluoromethoxybutane and nonafluoroethoxyisobutane; derivatives of perfluoromorpholine, such as 4-trifluoromethyl perfluoromorpholine sold under the name “PF 5052®” by the company 3M;

The wax or waxes may be selected from carnauba wax, candelilla wax, and Alpha wax, paraffin wax, ozokerite, the vegetable waxes such as olive wax, rice wax, hydrogenated jojoba wax or the absolute waxes of flowers such as the essential wax of blackcurrant flower sold by the company BERTIN (France), animal waxes such as beeswax, or modified beeswax (cerabellina); other waxes or waxy raw materials that can be used according to the present disclosure include the marine waxes such as that sold by the company SOPHIM under reference M82, the waxes of polyethylene or of polyolefins.

The saturated or unsaturated fatty acids may be selected from myristic acid, palmitic acid, stearic acid, behenic acid, oleic acid, linoleic acid, linolenic acid and isostearic acid.

The fatty esters may be chosen from the esters of carboxylic acids, for example, the mono-, di-, tri- or tetracarboxylates.

The esters of carboxylic acids may be chosen from the esters of saturated or unsaturated, linear or branched C₁-C₂₆ aliphatic acids and of saturated or unsaturated, linear or branched C₁-C₂₆ aliphatic alcohols, the total number of carbons in the esters being greater than or equal to 10.

Among the monoesters, we may mention, unlimitedly, dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; myristyl stearate; octyl isononanoate; 2-ethylhexyl isononate; octyl palmitate; octyl pelargonate; octyl stearate; octyidodecyl erucate; oleyl erucate; ethyl and isopropyl palmitates, ethyl-2-hexyl palmitate, 2-octyidecyl palmitate, alkyl myristates such as isopropyl, butyl, cetyl, 2-octyldodecyl myristate, hexyl stearate, butyl stearate, isobutyl stearate; dioctyl malate, hexyl laurate, 2-hexyldecyl laurate.

It is also possible to use the esters of C₄-C₂₂ di- or tricarboxylic acids and of C₁-C₂₂ alcohols and the esters of mono-, di- or tricarboxylic acids and of C₂-C₂₆ di-, tri-, tetra- or pentahydroxy alcohols.

Non-limiting mention may also be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisotearyl citrate; glyceryl trilactate; glyceryl trioctanoate; trioctyldodecyl citrate; trioleyl citrate; propylene glycol dioctanoate and neopentyl glycol diheptanoate; the esters mentioned above being different from the esters of formula (I).

Among the esters mentioned above, non-limiting mention may be made of the ethyl and isopropyl palmitates, ethyl-2-hexyl palmitate, 2-octyldecyl palmitate, alkyl myristates such as isopropyl, butyl, cetyl, 2-octyldodecyl myristate, hexyl stearate, butyl stearate, isobutyl stearate; dioctyl malate, hexyl laurate, 2-hexyidecyl hexyl and isononyl isononanate, cetyl octanoate.

Non-limiting examples of fatty alcohols include: saturated or unsaturated, linear or branched fatty alcohols having from 8 to 26 carbon atoms, such as cetyl alcohol, stearyl alcohol and their mixture (cetylstearyl alcohol), octyldodecanol, 2-butyloctanol, 2-hexyldecanol, 2-undecylpentadecanol, oleic alcohol or linoleic alcohol.

The fatty substances may be represent in an amount ranging from 0.1 to 50%; for instance, ranging from 1 to 30%, or ranging from 2 to 20 wt. % of the total composition.

As stated previously, the compositions can include at least one additional fixing polymers different from the polymers of the present disclosure. Fixing polymer means, for the purpose of the present disclosure, any polymer for imparting shape to the hair or for maintaining the hair in a given shape.

All the anionic, cationic, amphoteric and non-ionic fixing polymers, different from the polyurethanes (A) as well as mixtures thereof used in the industry can be used as additional fixing polymers in the compositions according to the present application.

The fixing polymers can be soluble in the cosmetically acceptable medium or insoluble in said medium and used in this case in the form of dispersions of solid or liquid particles of polymer (latex or pseudolatex).

The anionic fixing polymers may be selected from polymers having groups derived from carboxylic, sulphonic or phosphoric acid and have a number-average molecular weight ranging from 500 to 5,000,000.

The carboxyl groups are supplied by unsaturated mono- or dicarboxylic acid monomers such as those corresponding to the formula:

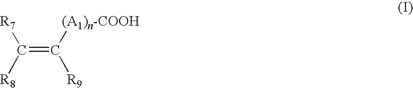

wherein n is an integer ranging from 0 to 10; A₁ is chosen from methylene groups, optionally joined to the carbon atom of the unsaturated group or to the adjacent methylene group when n is greater than 1, via a heteroatom such as oxygen or sulphur; R₇ is chosen from a hydrogen atom, phenyl groups, and benzyl groups; R₈ is chosen from a hydrogen atom, lower alkyl groups and carboxyl groups; R₉ is chosen from a hydrogen atom, lower alkyl groups, —CH₂—COOH, phenyl groups, and benzyl groups.

In the aforementioned formula, a lower alkyl group may be chosen from groups having 1 to 4 carbon atoms such as the methyl and ethyl groups.

The anionic fixing polymers with carboxyl groups according to the present disclosure may include, for example: A) The homo- or copolymers of acrylic or methacrylic acid or their salts, for example, the products sold under the names VERSICOL® E or K by the company ALLIED COLLOID and ULTRAHOLD® by the company BASF, the copolymers of acrylic acid and of acrylamide sold in the form of their sodium salts under the names RETEN 421, 423 or 425 by the company HERCULES, the sodium salts of the polyhydroxycarboxylic acids;

B) The copolymers of acrylic or methacrylic acid with a monoethylenic monomer such as ethylene, styrene, vinyl esters, esters of acrylic or methacrylic acid, optionally grafted on a polyalkylene glycol such as polyethylene glycol, and optionally crosslinked. Such polymers may be the ones described in French Patent No. 1 222 944 and German Patent Application No. 2 330 956, the copolymers of this type having in their chain an acrylamide unit optionally N-alkylated and/or hydroxyalkylated, as described in Luxembourg Patent Application Nos. 75370 and 75371 or offered under the name QUADRAMER by the company AMERICAN CYANAMID. Non-limiting mention may also be made of the acrylic acid/ethyl acrylate/N-tert-butylacrylamide terpolymers such as ULTRAHOLD STRONG sold by the company BASF. We may also mention the copolymers of acrylic acid and of C₁-C₄ alkyl methacrylate and the terpolymers of vinyl pyrrolidone, acrylic acid and C₁-C₂₀ alkyl methacrylate, for example lauryl methacrylate, such as that marketed by the company ISP under the name ACRYLIDONE® LM and the methacrylic acid/ethyl acrylate/tert-butyl acrylate terpolymers such as the product marketed under the name LUVIMER® 100 P by the company BASF.

We may also mention the methacrylic acid/acrylic acid/ethyl acrylate/methyl methacrylate copolymers in aqueous dispersion, marketed under the name AMERHOLD® DR 25 by the company AMERCHOL;

C) The copolymers of crotonic acid, such as those having vinyl acetate or propionate units in their chain, and optionally other monomers such as the allyl or methallyl esters, vinyl ether or vinyl ester of a saturated, linear or branched carboxylic acid, with long hydrocarbon chain, such as those having at least 5 carbon atoms, and said polymers can optionally be grafted or crosslinked, or alternatively another vinyl, allyl or methallyl ester monomer of an α- or β-cyclic carboxylic acid. Such polymers may be the ones described in, among others, French Patent Nos. 1 222 944,1 580 545, 2 265 782, 2 265 781 1 564 110 and 2 439 798. Commercial products included in this class are the resins 28-29-30, 26-13-14 and 28-13-10 marketed by the company National Starch;

D) The copolymers of C₄-C₈ monounsaturated carboxylic acids or anhydrides selected from:
copolymers comprising (i) at least one group chosen from maleic, fumaric, itaconic acids and anhydrides and (ii) at least one monomer selected from vinyl esters, vinyl ethers, vinyl halides, phenylvinylic derivatives, acrylic acid and its esters. Anhydride functions of these copolymers being optionally monoesterified or monoamidated. Such polymers may be the ones described in U.S. Pat. No. 2,047,398, U.S. Pat. No 2,723,248, U.S. Pat. No. 2,102,113, Great Britain Patent No. 839 805. Commercial products may include those sold under the names GANTREZ® AN or ES by the company ISP;
copolymers comprising (i) at least one unit chosen from maleic, citraconic, itaconic anhydride units and (ii) at least one monomer selected from allyl or methallyl esters optionally with at least one group chosen from acrylamide, methacrylamide, α-olefin groups, acrylic esters, methacrylic esters, acrylic acids, methacrylic acids, and vinyl pyrrolidone in their chain,

the anhydride functions of these copolymers being optionally monoesterified or monoamidated.

These polymers are for example described in French Patent Nos. 2 350 384 and 2 357 241;

E) Polyacrylamides having carboxylate groups.

F) Homopolymers and copolymers containing sulphonic groups such as the polymers having vinylsulphonic, styrene-sulphonic, naphthalene-sulphonic or acrylamido-alkylsulphonic units different from the branched sulphonic polyesters of the present disclosure.

These polymers may be selected from:
salts of polyvinylsulphonic acid having a molecular weight between about 1000 and 100,000, as well as copolymers with an unsaturated comonomer such as acrylic or methacrylic acids and their esters, as well as acrylamide or its derivatives, vinyl ethers and vinyl pyrrolidone;
salts of polystyrene-sulphonic acid such as the sodium salts sold for example under the name FLEXAN® 500 and FLEXAN® 130 by National Starch. These compounds may be the ones described in patent FR 2 198 719;
salts of polyacrylamide-sulphonic acids such as those mentioned in patent US 4 128 631, for example, the polyacrylamidoethylpropane-sulphonic acid sold under the name COSMEDIA POLYMER HSP 1180 by HENKEL.

As another anionic fixing polymer that can be used according to the present disclosure, we may mention the branched anionic block polymer sold under the name FIXATE™ G-100 Polymer by the company LUBRIZOL.

According to the present disclosure, the anionic fixing polymers may be selected from the copolymers of acrylic acid or of acrylic esters such as the acrylic acid/ethyl acrylate/N-tert-butylacrylamide terpolymers sold under the name ULTRAHOLD® STRONG by the company BASF, copolymers derived from crotonic acid such as the vinyl acetate/vinyl tert-butylbenzoate/crotonic acid terpolymers and the crotonic acid/vinyl acetate/vinyl neododecanoate terpolymers sold under the name Resin 28-29-30 by the company NATIONAL STARCH, the polymers derived from maleic, fumaric, itaconic acids or anhydrides with vinyl esters, vinyl ethers, vinyl halides, phenylvinyl derivatives, acrylic acid and its esters such as the monoesterified methylvinylether/maleic anhydride copolymers sold for example under the name GANTREZE® by the company ISP, the copolymers of methacrylic acid and methyl methacrylate sold under the name EUDRAGI® L by the company RÖHM PHARMA, the copolymers of rnethacrylic acid and ethyl acrylate sold under the name LUVIMER® MAEX or MAE by the company BASF and the vinyl acetate/crotonic acid copolymers sold under the name LUVISET CA 66 by the company BASF and the vinyl acetate/crotonic acid copolymers grafted with polyethylene glycol sold under the name ARISTOFLE® A by the company BASF, and the polymer sold under the name FIXATE™ G-100 Polymer by the company LUBRIZOL.

Among the anionic fixing polymers mentioned above, within the scope of the present disclosure, non-limiting mention may be made of the monoesterified methylvinyl ether/maleic anhydride copolymers sold under the name GANTREZ® ES 425 by the company ISP, the acrylic acid/ethyl acrylate/N-tert-butylacrylamide terpolymers sold under the name ULTRAHOLD® STRONG by the company BASF, the copolymers of rnethacrylic acid and methyl methacrylate sold under the name EUDRAGIT® L by the company ROHM PHARMA, the vinyl acetate/vinyl tert-butylbenzoate/crotonic acid terpolymers and the crotonic acid/vinyl acetate/vinyl neododecanoate terpolymers sold under the name RESIN 28-29-30 by the company NATIONAL STARCH, the copolymers of methacrylic acid and ethyl acrylate sold under the name LUVIMER® MAEX or MAE by the company BASF, the vinyl pyrrolidone/acrylic acid/lauryl methacrylate terpolymers sold under the name ACRYLIDONE® LM by the company ISP, the polymer sold under the name FIXATE™ G-100 Polymer by the company LUBRIZOL.

The cationic film-forming fixing polymers for use according to the present disclosure may be selected from the polymers having primary, secondary, tertiary and/or quaternary amine groups forming part of the polymer chain or joined directly to the latter, and having a molecular weight ranging from 500 to about 5,000,000 such as ranging from 1000 to 3,000,000.

Among these polymers, non-limiting mention may be made of the following catioric polymers:

(1) the homopolymers or copolymers derived from acrylic or methacrylic esters or amides and having at least one of the units with the following formulae:

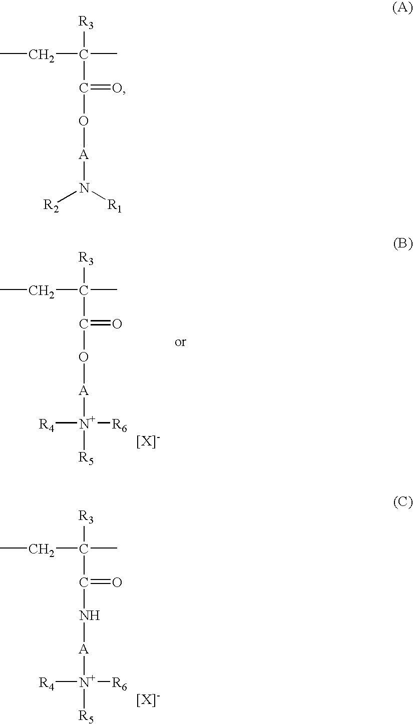

wherein:

R₃ is chosen from a hydrogen atom and CH₃ radicals;

A is chosen from linear or branched alkyl groups having from 1 to 6 carbon atoms and hydroxyalkyl groups having from 1 to 4 carbon atoms;

R₄, R₅, R₆, which may be identical or different, are chosen from alkyl groups having of 1 to 18 carbon atoms and benzyl radicals;

R₁ and R₂, which may be identical or different, are chosen from a hydrogen atom and alkyl groups having from 1 to 6 carbon atoms;

X is chosen from methosulphate anions and halides such as chloride or bromide.

The copolymers of family (1) additionally contain at least one unit derived from comonomers, which can be selected from the family comprising the acrylamides, methacrylamides, diacetone-acrylamides, acrylamides and methacrylamides substituted on the nitrogen with lower alkyl groups (C1-C4), groups derived from acrylic or methacrylic acids or their esters, vinyl lactams such as vinyl pyrrolidone or vinyl caprolactam, vinyl esters.

Thus, among these copolymers of family (1), we may mention, for example:
the copolymers of acrylamide and dimethylaminoethyl methacrylate quaternized with dimethyl sulphate or with a dimethyl halide, such as that sold under the name HERCOFLOC® by the company HERCULES,
the copolymers of acrylamide and methacryloyloxyethyltrimethylammonium chloride described for example in European Patent Application No. A-080976 and sold under the name BINA QUAT P 100 by the company CIBA GEIGY,
the copolymer of acrylamide and methacryloyloxyethyltrimethylammonium methosulphate such as that sold under the name RETEN by the company HERCULES,
the vinyl pyrrolidone/dialkylaminoalkyl acrylate or methacrylate copolymers, quaternized or unquaternized, such as the products sold under the name “GAFQUAT® “by the company ISP for example “GAFQUAT® 734” or “GAFQUAT® 755” or else the products called “COPOLYMER 845, 958 and 937”. These polymers are described in detail in French Patent Nos. 2 077 143 and 2 393 573,
the fatty-chain polymers with a vinyl pyrrolidone unit, such as the products sold under the name STYLEZE W20 and STYLEZE W10 by the company ISP,
the dimethylaminoethyl methacrylate/vinyl caprolactam/vinyl pyrrolidone terpolymers such as the product marketed under the name GAFFIX VC 713 by the company ISP, and
the quaternized vinyl pyrrolidone/dimethylaminopropyl methacrylamide copolymers such as the products marketed under the name “GAFQUAT® HS 100” by the company ISP;

(2) the non-cellulosic cationic polysaccharides, for example, the non-cellulosic cationic polysaccharides with quaternary ammonium such as those described in U.S. Pat. Nos. 3,589,578 and 4,031,307 such as the guar gums containing cationic trialkylammonium groups. For instance, the products marketed under the trade names JAGUAR C13 S, JAGUAR C 15, JAGUAR C 17 by the company MEYHALL.

(3) the quaternary copolymers of vinyl pyrrolidone and vinyl imidazole;

(4) the chitosans or their salts; the salts that can be used include the acetate, lactate, glutamate, gluconate or pyrrolidone-carboxylate of chitosan.

Among these compounds, we may mention chitosan having a degree of deacetylation of 90.5 wt. % sold under the name KYTAN BRUT STANDARD by the company ABER TECHNOLOGIES, the chitosan pyrrolidone-carboxylate marketed under the name KYTAMER® PC by the company AMERCHOL.

(5) the cationic derivatives of cellulose such as the copolymers of cellulose or of cellulose derivatives grafted with a water-soluble monomer bearing a quaternary ammonium, and described in, for example, U.S. Pat. No. 4,131,576, for instance the hydroxyalkyl celluloses, such as the hydroxymethyl, hydroxyethyl or hydroxypropyl celluloses grafted with a methacryloyloxyethyl trimethylammonium salt, methacrylamidopropyl trimethylammonium salt, or dimethyl-diallylammonium salt.

The commercial products corresponding to this definition include, for example, the products sold under the name “CELQUAT L 200” and “CELQUAT H 100” by the company National Starch.

The amphoteric fixing polymers that can be used according to the present disclosure can be selected from the polymers having units B and C randomly distributed in the polymer chain, wherein B is chosen from units derived from a monomer having at least one basic nitrogen atom and C is chosen from units derived from an acidic monomer having one or more carboxyl or sulphonic groups, or else B and C may be chosen from groups derived from zwitterionic monomers of carboxybetaines or sulphobetaines;

B and C may also be chosen from cationic polymer chains having primary, secondary, tertiary or quaternary amine groups, wherein at least one of the amine groups bears a carboxyl or sulphonic group joined via a hydrocarbon group, or else B and C form part of a chain of a polymer with an ethylene-α,β-dicarboxyl unit in which one of the carboxyl groups has been reacted with a polyamine bearing one or more primary or secondary amine groups.

As non-limiting examples, amphoteric fixing polymers corresponding to the definition given above may be selected from the following polymers:

(1) the copolymers with acidic vinyl units and with basic vinyl units, such as those resulting from the copolymerization of a monomer derived from a vinylic compound bearing a carboxyl group such as acrylic acid, methacrylic acid, maleic acid, alpha-chloroacrylic acid, and of a basic monomer derived from a substituted vinylic compound containing at least one basic atom, such as dialkylaminoalkyl methacrylate and acrylate, dialkylaminoalkylmethacrylamide and acrylamide. Such compounds may be the ones described in U.S. Pat. No. 3,836,537.

(2) the polymers having units derived from:

a) at least one monomer selected from the acrylamides or the methacrylamides substituted on the nitrogen atom with an alkyl group,

b) at least one acidic comonomer containing one or more reactive carboxyl groups, and

c) at least one basic comonomer such as esters with primary, secondary, tertiary and quaternary amine substituents of acrylic and methacrylic acids, and the product of quaternization of dimethylaminoethyl methacrylate with dimethyl or diethyl sulphate.

The N-substituted acrylamides or methacrylamides, according to the present disclosure, may be chosen from compounds in which the alkyl groups have from 2 to 12 carbon atoms, for example, N-ethylacrylamide, N-tert-butylacrylamide, N-tert-octylacrylamide, N-octylacrylamide, N-decylacrylamide, N-dodecylacrylamide as well as the corresponding methacrylamides.

The acidic comonomers may be selected from acrylic, methacrylic, crotonic, itaconic, maleic, fumaric acids as well as the alkyl monoesters having 1 to 4 carbon atoms of maleic or fumaric acids or anhydrides.

The basic comonomers include, as a non-limiting example, aminoethyl, butylaminoethyl, N,N′-dimethylaminoethyl, N-tert-butylaminoethyl methacrylates.

The copolymers may include those whose CTFA designation (4th Ed., 1991) is octylacrylamide/acrylates/butylaminoethyl-methacrylate copolymer, such as the products sold under the name AMPHOMER® or LOVOCRYL® 47 by the company NATIONAL STARCH.

(3) the crosslinked polyaminoamides acylated partially or completely, derived from polyaminoamides of general formula:

CO—R₁₀—CO-Z (II)

wherein R₁₀ is chosen from divalent groups derived from a saturated dicarboxylic acid, from an aliphatic mono- or dicarboxylic acid with ethylenic double bond, from an ester of a lower alkanol having 1 to 6 carbon atoms of these acids or from a group derived from the addition of any one of said acids with a bis-primary or bis-secondary amine, and Z is chosen from groups derived from a bis-primary, mono- or bis-secondary polyalkylene-polyamine, for instance, Z may be chosen from:

a) in the proportions from 60 to 100 mol. %, the group

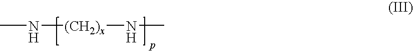

wherein x=2 and p=2 or 3, or else x=3 and p=2

this group being derived from diethylenetriamine, triethylenetetramine or dipropylenetriamine;

b) in the proportions from 0 to 40 mol. %, the above group (III), wherein x=2 and p=1 and is derived from ethylenediamine, or the group derived from piperazine:

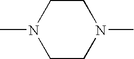

c) in the proportions from 0 to 20 mol. %, the group —NH—(CH₂)₆—NH— derived from hexamethylenediamine,

these polyaminoamides being crosslinked by a reaction of addition of a bifunctional crosslinking agent selected from the epihalohydrins, diepoxides, dianhydrides, bis-unsaturated derivatives, using 0.025 to 0.35 mol of crosslinking agent per amine group of the polyaminoamide, and acylated by the action of acrylic acid, chloroacetic acid or an alkane-sultone or salts thereof.

The saturated carboxylic acids may be selected from the acids having 6 to 10 carbon atoms such as adipic, trimethyl-2,2,4-adipic and trimethyl-2,4,4-adipic, terephthalic acids, acids with an ethylenic double bond, for example, acrylic, methacrylic, itaconic acids.

The alkane-sultones used in the acylation may include propane- or butane-sultone, the salts of the acylating agents such as the sodium or potassium salts.

(4) the polymers having zwitterionic units of formula:

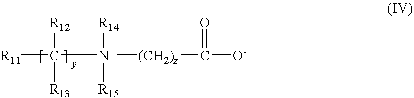

wherein R₁₁ is chosen from a polymerizable unsaturated group such as an acrylate, methacrylate, acrylamide or methacrylamide group, y and z are chosen from an integer ranging from 1 to 3, R₁₂ and R₁₃ are chosen from a hydrogen atom, methyl groups, ethyl groups, and propyl groups, R₁₄ and R₁₅ are chosen from a hydrogen atom and alkyl groups in such a way that the sum of the carbon atoms in R₁₄ and R₁₅ is less than or equal to 10.

The polymers containing such units can also have units derived from non-zwitterionic monomers such as dimethyl- or diethylaminoethyl acrylate or methacrylate or alkyl acrylates or methacrylates, acrylamides or methacrylamides, or vinyl acetate.

As non-limiting examples, methyl methacrylatelmethyl dimethyl-carboxy-methylammonio-ethylmethacrylate copolymers, such as the product sold under the name DIAFORMER Z301 by the company SANDOZ.

(5) the polymers derived from chitosan having monomer units corresponding to the following formulae:

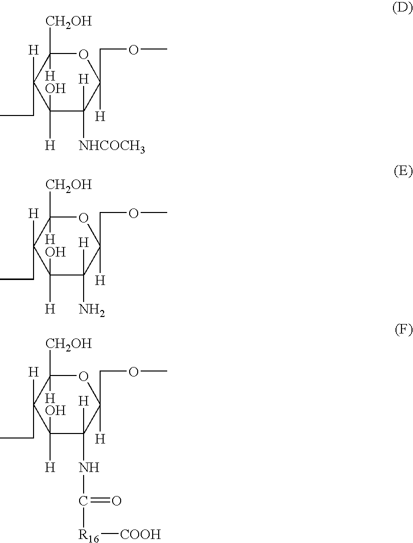

unit (D) being present in proportions between 0 and 30%, unit (E) in proportions between 5 and 50% and unit (F) in proportions between 30 and 90%, it being understood that in this unit (F), R₁₆ represents a group of formula:

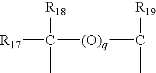

wherein, if q=0, R₁₇, R₁₈ and R₁₉, which may be identical or different, are chosen from a hydrogen atom, methyl, hydroxyl, acetoxy, amino, monoalkylamine, and dialkylamine residues optionally interrupted by at least one nitrogen atom and/or optionally substituted with at least one residure chosen from amine, hydroxyl, carboxyl, alkylthio, sulphonic, and alkylthio residues, wherein the alkyl group bears an amino residue, at least one of the groups R₁₇, R₁₈ and R₁₉ being in this case a hydrogen atom;

or if q=1, R₁₇, R₁₈ and R₁₉ are chosen from a hydrogen atom and salts formed by these compounds with bases or acids.

(6) The polymers with the units corresponding to general formula (XII) are, for example, described in French Patent No. 1 400 366:

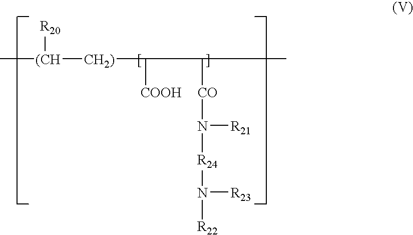

wherein R₂₀ is chosen from a hydrogen atom, CH₃O, CH₃CH₂O, and phenyl groups, R₂₁ is chosen from a hydrogen atom and lower alkyl groups such as methyl, ethyl, R₂₂ is chosen from a hydrogen atom and C₁-C₆ lower alkyl groups such as methyl, ethyl, R₂₃ is chosen from C₁-C₆ lower alkyl groups such as methyl, ethyl and groups corresponding to the formula: —R₂₄—N(R₂₂)₂, R₂₄ is chosen from —CH₂—CH₂—, —CH₂—CH₂—CH₂—, —CH₂—CH(CH₃)—, R₂₂ having the meanings given above.

(7) The polymers derived from the N-carboxyalkylation of chitosan such as N-carboxymethyl-chitosan or N-carboxybutyl-chitosan, sold under the name “EVALSAN” by the company JAN DECKER.

(8) The amphoteric polymers of the type -D-X-D-X selected from:

a) the polymers obtained by the action of chloroacetic acid or sodium chloroacetate on compounds having at least one unit of formula:

-D-X-D-X-D- (VI)

wherein D is chosen from groups of:

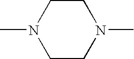

and X denotes the symbol E or E′. E or E′, which may be identical or different, are chosen from divalent groups, which are linear or branched alkylene groups, with up to 7 carbon atoms in the main chain, unsubstituted or substituted with hydroxyl groups and which can additionally comprise oxygen, nitrogen, or sulphur atoms, 1 to 3 aromatic and/or heterocyclic rings; the oxygen, nitrogen and sulphur atoms being present in the form of ether, thioether, sulphoxide, sulphone, sulphonium, alkylamine, alkenylamine groups; hydroxyl, benzylamine, amine oxide, quaternary ammonium, amide, imide, alcohol, ester and/or urethane groups.

b) The polymers of formula:

-D-X-D-X- (VII)

wherein D is chosen from groups of:

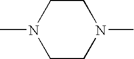

and X denotes the symbol E or E′ and at least once E′; E having the meaning stated above and E′ is a divalent group, which is a linear or branched alkylene group, with up to 7 carbon atoms in the main chain, unsubstituted or substituted with one or more hydroxyl groups and bearing one or more nitrogen atoms, the nitrogen atom being substituted with an alkyl chain interrupted optionally by an oxygen atom and which must have one or more carboxyl functions or one or more hydroxyl functions, betainized by reaction with chloroacetic acid or sodium chloroacetate.

(9) the alkyl(C₁-C₅)vinyl ether/maleic anhydride copolymers partially modified by semiamidation with an N,N-dialkylaminoalkylamine such as N,N-dimethylamino-propylamine or by semiesterification with an N,N-dialkylaminoalkanol. These copolymers can also contain other vinyl comonomers such as vinyl caprolactam.

In at least one embodiment, the at least one amphoteric fixing polymer is chosen from those of family (3) such as the copolymers whose CTFA designation is octylacrylamide/acrylates/butylamino-ethylmethacrylate copolymer, such as the products sold under the names AMPHOMER®, AMPHOMER® LV 71 or LOVOCRYL® 47 by the company NATIONAL STARCH and those of family (4) such as the methyl methacrylate/methyl dimethyl-carboxymethylammonio-ethylmethacrylate copolymers sold for example under the name DIAFORMER Z301 by the company SANDOZ.

The non-ionic fixing polymers for use according to the present disclosure are selected, for example, from:
the polyalkyloxazolines;
the homopolymers of vinyl acetate;
the copolymers of vinyl acetate such as, for example, copolymers of vinyl acetate and acrylic ester, copolymers of vinyl acetate and ethylene, or copolymers of vinyl acetate and maleic ester, for example, dibutyl maleate;
the homopolymers and copolymers of acrylic esters such as, for example, the copolymers of alkyl acrylates and of alkyl methacrylates such as the products offered by the company RÖHM & HAAS under the names PRIMAL® AC-261 K and EUDRAGIT® NE 30 D, by the company BASF under the name 8845, by the company HOECHST under the name APPRETAN® N9212;
the copolymers of acrylonitrile and of a non-ionic monomer selected for example from butadiene and alkyl (meth)acrylates; we may mention the products offered under the name CJ 0601 B by the company RÖHM & HAAS;
the homopolymers of styrene;
the copolymers of styrene, for example the copolymers of styrene and alkyl (meth)acrylate such as the products MOWILITH® LDM 6911, MOWILITH® DM 611 and MOWILITH® LDM 6070 offered by the company HOECHST, the products RHODOPAS® SD 215 and RHODOPAS® DS 910 offered by the company RHONE POULENC; the copolymers of styrene, of alkyl methacrylate and of alkyl acrylate; the copolymers of styrene and butadiene; or the copolymers of styrene, butadiene and vinylpyridine;
the polyamides;
the homopolymers of vinyl lactam such as the homopolymers of vinyl pyrrolidone and such as the polyvinyl caprolactam marketed under the name LUVISCOL® PLUS by the company BASF; and
the copolymers of vinyl lactam such as a poly(vinyl pyrrolidone/vinyl lactam) copolymer sold under the trade name LUVITEC® VPC 55K65W by the company BASF, the poly(vinyl pyrrolidone/vinyl acetate) copolymers such as those marketed under the name PVPVA® S630L by the company ISP, Luviscol® VA 73, VA 64, VA 55, VA 37 and VA 28 by the company BASF; and the poly(vinyl pyrrolidone/vinyl acetate/vinyl propionate) terpolymers, for example that marketed under the name LUVISCOL® VAP 343 by the company BASF.

The alkyl groups of the non-ionic polymers mentioned above may be chose from the alkyls groups comprising from 1 to 6 carbon atoms.

According to the present disclosure, use can also be made of fixing polymers of the grafted silicone type, comprising a polysiloxane part and a part constituted of a non-silicone organic chain, one of the two parts constituting the main chain of the polymer and the other being grafted onto said main chain.

These polymers are for example described in European Patent Application Publication Nos.: EP-A-0 412 704, EP-A-0 412 707, EP-A-0 640 105, and EP-A-0 582 152, and International Patent Application Publication Nos.: WO 95/00578 and WO 93/23009, and U.S. Pat. Nos. 4,693,935; 4,728,571; and 4,972,037.

These polymers can be amphoteric, anionic or non-ionic, for example, anionic polymers or non-ionic polymers.

Such polymers are, for example, the copolymers that are obtainable by radical polymerization starting from a mixture of monomers comprising:

a) from 50 to 90 wt. % of tert-butyl acrylate,

b) from 0 to 40 wt. % of acrylic acid,

c) from 5 to 40 wt. % of a silicone macromer of formula

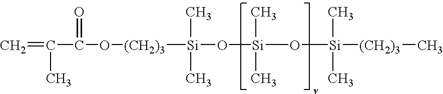

wherein v is a number ranging from 5 to 700, the percentages by weight being calculated relative to the total weight of the monomers.

Other examples of grafted silicone polymers include, but not limited to, polydimethylsiloxanes (PDMS) onto which mixed polymer units of the poly(meth)acrylic acid type and of the poly(alkyl(meth)acrylate) type are grafted via a joining group of the thiopropylene type, and polydimethylsiloxanes (PDMS) onto which polymer units of the poly(isobutyl(meth)acrylate) type are grafted via a joining group of the thiopropylene type.

As another type of silicone fixing polymers, we may mention the product LUVIFLEX® SILK marketed by the company BASF.

Polyurethanes different from the polyurethane (A), functionalized or unfunctionalized, siliconized or unsiliconized, cationic, non-ionic, anionic or amphoteric, or mixtures thereof, can also be used as fixing polymers.

The polyurethanes may include, as non-limiting examples, the ones described in European Patent Application Publication Nos.: EP 0 751 162, EP 0 637 600, and EP 0 648 485, and French Patent No. 2 743 297, as well as in European Patent Application Publication No. EP 0 656 021 and Internation Patent Application Publication No. WO 94/03510 of the company BASF, and European Patent Applicantion Publication No. EP 0 619 111 of the company National Starch.

Polyurethanes that may be suitable for the present disclosure also include, as non-limiting examples, the products marketed under the names LUVISET PURE® and LUVISET® Si PURE by the company BASF.

The concentration of additional fixing polymer(s) used in the compositions according to the present disclosure may range from 0.1 to 20%, for instance, range from 0.5 and 10 wt. % relative to the total weight of the composition.

A person skilled in the art would be able to add the additives without disturbing the properties of the compositions of the present disclosure.

As a non-limiting example, the compositions are in the form of gels. For instance, these compositions may be transparent or translucent. For example, they may have a viscosity greater than 500 cP at a temperature of 25° C. and at a shear rate of 1 s⁻¹.

In at least one embodiment, the compositions according to the present disclosure, their turbidity at 25° C., measured with a HACH Model 2100P turbidimeter, may range from 0 to 900 NTU units, such as range from 0 to 500 NTU units.

The pH of the formulas associated with the present disclosure ranges from 1 to 13, for instance, ranges from 3 to 11, such as ranges from 6 and 9.

When the composition according to the present disclosure is packaged in an aerosol device, it includes at least one propellant, which can be selected from the volatile hydrocarbons, such as N-butane, isobutane, pentane, the halogenated hydrocarbons, dimethyl ether (DME) and mixtures thereof. Carbon dioxide, nitrous oxide, nitrogen, or compressed air can also be used as propellant. Mixtures of propellants can also be used. For example, dimethyl ether is used.

The propellant may be present in an amount ranging from 5 to 90 wt. % relative to the total weight of the composition in the aerosol device, and, for example, in an amount ranging from 10 to 60%.

The composition according to the present disclosure may be used in non-rinse application on the hair.

The present disclosure also provides a method of shaping keratin fibers such as hairs, comprising: applying a cosmetic composition according to the present disclosure. In addition, the present disclosure also provides a method of styling keratin fibers such as hairs, comprising: applying a composition according to the present disclosure to the hair, optionally rinsing the hair, then shaping and drying the hair.

Notwithstanding that the numerical ranges and parameters setting forth the broad scope are approximations, the numerical values set forth in the specific examples are reported as precisely as possible. Any numerical value, however, inherently contain certain errors necessarily resulting from the standard deviation found in their respective testing measurements. By way of non-limiting illustration, a concrete example of preparation of the composition according to the present disclosure are given below.

### EXAMPLES

In the following tables, the values are expressed in percentages by weight of active substance in the final formula.

The viscosity measurements were performed with a RHEOMAT RM180 rheometer from the company Mettler, with a speed gradient of 200 rev/min (spindle 4).

The turbidity was measured with the HACH turbidimeter—Model 2100 P at 25° C. in NTU units (NTU: nephelometric turbidity units).

The elastomeric anionic film-forming polyurethanes (A) used in the examples are defined below on the basis of their chemical composition and mechanical properties. The compositions of the elastomeric anionic film-forming polyurethanes (A) are expressed in dry matter.

| | Elongation | Instantaneous | Recovery |
|---|---|---|---|
| | at break | recovery | at 300 s |
| A1 | 1300 | 70.5 | 90.6 |
| A2 | 833 | 74.9 | 90.8 |
| A3 | >1200 | 84.2 | 94.2 |
| A4 | | 77.6 | 93.5 |
| A5 | | 76.7 | 94.6 |
| A6 | 877 | 74.7 | 91.8 |

| | IPDI | PEO | PPG | DMPA | TMP | HNA | PDMS | MPEG | EDA | TEA | NaOH | TOTAL |
|---|---|---|---|---|---|---|---|---|---|---|---|---|
| A1 | 28.1 | 22.7 | 26.3 | 3.0 | 0.5 | 14.5 | | | 2.7 | 2.3 | | 100.0 |
| A2 | 28.9 | 16.7 | 23.0 | 2.9 | 0.5 | 23.0 | | | 2.7 | 2.1 | | 100.0 |
| A3 | 25.9 | 16.7 | 48.5 | 3.0 | | | | | 3.2 | 2.7 | | 100.0 |
| A4 | 23.8 | 15.3 | 44.4 | 2.8 | | | | 8.3 | 2.9 | 2.5 | | 100.0 |
| A5 | 24.6 | 15.9 | 46.4 | 2.9 | 1.6 | | 3.0 | | 3.0 | 0.9 | 1.6 | 100.0 |
| A6 | 29.1 | 14.1 | 49.7 | 2.9 | 0.5 | | | | 2.3 | 1.1 | 0.5 | 100.0 |

For all the polyurethanes A1 to A6, the ethylene oxide side chains have a molecular weight of 1200 g/mol and they represent more than 12 wt. % of the polyurethane (A).

The thickening polymers used in the examples are as follows:

Polymer 1: Jaguar HP 105, supplied by the company Rhodia.

Polymer 2: Rheolate FX 1100, supplied by the company Elementis.

Polymer 3: Dermothix 100, supplied by the company Alzo.

Polymer 4: Aculyn 46, supplied by the company Rohm and Haas.

Polymer 5. Aculyn 44, supplied by the company Rohm and Haas.

Polymer 6: Klucel EF Pharma, supplied by the company Aqualon.

Polymer 7: Pemulen® Polymeric Emulsifier, supplied by the company Lubrizol.

Polymer 8: Synthalen K, supplied by the company 3V.

Polymer 9: Viscophobe DB 1000, supplied by the company Amerchol

IPDI: Isophorone Diisocyanate

HNA: He,xane Neopentyl adipate polyester polyol (Mw=1000)

PPG: Polypropylene glycol (Mw=2000)

PEO: Polyethylene oxide-1,3-diol (Mw=1200)

DMPA: Dimethylolpropionic acid

TMP: Trimethylolpropane

EDA: Ethylenediamine

TEA: Triethylamine

PDMS: Polydimethylsiloxane polyol

MPEG: Methoxy Polyethylene Glycol

NaOH: Sodium hydroxide

EtOH: Ethanol

The appearance of the gel was assessed using the following notation:

1/ homogeneous gel, transparent/translucent and colourless,

2/ opalescent homogeneous gel,

3/ inhomogeneous gel, lumpy, opaque and white.

### Compositions of Gels Translucent to Transparent in Water, for Shaping the Hair:

| | 1 | 2 | 3 | 4 | 5 | 6 | 7 | 8 | 9 | 10 |
|---|---|---|---|---|---|---|---|---|---|---|
| Compositions | INVENTION | | | | | | | | | |
| A1 | 10 | 10 | 8 | 7 | 7 | 7 | 7 | 7 | 6 | 6 |
| Polymer 1: | 2 | — | — | — | — | — | — | — | 2 | — |
| Hydroxypropyl guar | | | | | | | | | | |
| Polymer 2: | — | 4 | — | — | — | — | — | — | — | 3.5 |
| Hexamethyl | | | | | | | | | | |
| diisocyanate/polyethylene | | | | | | | | | | |
| glycol with | | | | | | | | | | |
| alpha-omega | | | | | | | | | | |
| termination | | | | | | | | | | |
| Stearyl | | | | | | | | | | |
| polyoxyethylene | | | | | | | | | | |
| Polymer 3: | — | — | 3.5 | 3.5 | — | — | 3.5 | — | | |
| Polyethylene glycol- | | | | | | | | | | |
| 100 Stearyl ether | | | | | | | | | | |
| dimer | | | | | | | | | | |
| Isophorone | | | | | | | | | | |
| diisocyanate | | | | | | | | | | |
| Polymer 4: | | | | | 3 | — | — | — | | |
| Polyethylene glycol- | | | | | | | | | | |
| 150/Stearyl | | | | | | | | | | |
| alcohol/Methylene | | | | | | | | | | |
| diphenyl | | | | | | | | | | |
| diisocyanate | | | | | | | | | | |
| Polymer 5: | | | | | — | 5 | — | — | | |
| Polyethylene glycol- | | | | | | | | | | |
| 150/Decyl | | | | | | | | | | |
| alcohol/Methylene | | | | | | | | | | |
| diphenyl | | | | | | | | | | |
| diisocyanate | | | | | | | | | | |
| Polymer 6: | | | | | | | — | 15 | | |
| Hydroxypropylcellulose | | | | | | | | | | |
| Polymer 7: | — | — | — | — | — | — | — | — | | |
| Acrylate/C10-C30 | | | | | | | | | | |
| alkyl acrylate | | | | | | | | | | |
| Polymer 8: | | | | | | | | | | |
| carboxyvinylic | | | | | | | | | | |
| Polymer 9: | — | — | — | — | — | — | — | — | | |
| Polyacrylate-3 | | | | | | | | | | |
| Aminomethyl | — | — | — | — | — | — | — | — | | |
| propanol | | | | | | | | | | |
| Ethanol | 3.6 | 3.6 | 2.9 | 2.5 | 2.5 | 2.5 | 4.5 | Q.S. 100 | 2.1 | 2.1 |
| Distilled water | Q.s. 100 | Q.s. 100 | Q.s. 100 | Q.s. 100 | Q.s. 100 | Q.s. 100 | Q.s. 100 | 15.5 | Q.s. 100 | Q.s. 100 |
| Viscosity (cP) | 3140 | 16797 | 6055 | 4996 | 5960 | 7863 | 6852 | 6247 | 2958 | 9480 |
| Turbidity (NTU) | 693 | 192 | 209 | 103 | 658 | 852 | 103 | 186 | 231 | 135 |
| Appearance of gel | 2 | 1 | 1 | 1 | 2 | 2 | 1 | 1 | 1 | 1 |

| | 11 | 12 | 13 | 14 | 15 | 16 | 17 |
|---|---|---|---|---|---|---|---|
| Compositions | COMPARATIVE | | | | | | |
| A1 | 10 | 10 | 7 | 7 | 6 | 6 | 6 |
| Polymer 1: | — | — | — | — | | | |
| Hydroxypropyl guar | | | | | | | |
| Polymer 2: | — | — | — | — | | | |
| Hexamethyl | | | | | | | |
| diisocyanate/polyethylene | | | | | | | |
| glycol with | | | | | | | |
| alpha-omega | | | | | | | |
| termination | | | | | | | |
| Stearyl | | | | | | | |
| polyoxyethylene | | | | | | | |
| Polymer 3: | — | — | — | — | | | |
| Polyethylene glycol- | | | | | | | |
| 100 Stearyl ether | | | | | | | |
| dimer | | | | | | | |
| Isophorone | | | | | | | |
| diisocyanate | | | | | | | |
| Polymer 4: | — | — | — | — | | | |
| Polyethylene glycol- | | | | | | | |
| 150/Stearyl | | | | | | | |
| alcohol/Methylene | | | | | | | |
| diphenyl | | | | | | | |
| diisocyanate | | | | | | | |
| Polymer 5: | — | — | — | — | | | |
| Polyethylene glycol- | | | | | | | |
| 150/Decyl | | | | | | | |
| alcohol/Methylene | | | | | | | |
| diphenyl | | | | | | | |
| diisocyanate | | | | | | | |
| Polymer 6: | — | — | — | — | | | |
| Hydroxypropylcellulose | | | | | | | |
| Polymer 7: | 1.6 | | 1.6 | | 1.6 | — | — |
| Acrylate/C10-C30 | | | | | | | |
| alkyl acrylate | | | | | | | |
| Polymer 8: | | | | | — | 0.6 | — |
| carboxyvinylic | | | | | | | |
| Polymer 9: | | 0.7 | | 0.7 | — | — | 0.7 |
| Polyacrylate-3 | | | | | | | |
| Aminomethyl | QS pH7 | | | | | | |
| propanol | | | | | | | |
| Ethanol | 3.6 | 3.6 | 2.5 | 2.5 | 2.1 | 2.1 | 2.1 |
| Distilled water | Q.s. 100 | Q.s. 100 | Q.s. 100 | Q.s. 100 | Q.s. 100 | Q.s. 100 | Q.s. 100 |
| Viscosity (cP) | 3420 | 9521 | 2865 | 7136 | 2670 | 4080 | 6210 |
| Turbidity (NTU) | >1000 (not measurable, limit of detection of | | | | | | |
| | the apparatus) | | | | | | |
| Appearance of gel | 3 | 3 | 3 | 3 | 3 | 3 | 3 |

| | 18 | 19 | 20 | 21 | 22 | 23 | 24 | 25 | 26 |
|---|---|---|---|---|---|---|---|---|---|
| Compositions | INVENTION | | | | | | | | |
| A2 | 10 | 10 | 7 | 7 | 7 | 7 | 7 | 6 | 6 |
| Polymer 1: | 2 | — | — | — | — | — | — | 2 | — |
| Hydroxypropyl guar | | | | | | | | | |
| Polymer 2: | — | 4 | — | — | — | — | — | — | 3.5 |
| Hexamethyl | | | | | | | | | |
| diisocyanate/polyethylene | | | | | | | | | |
| glycol with | | | | | | | | | |
| alpha-omega | | | | | | | | | |
| termination | | | | | | | | | |
| Stearyl | | | | | | | | | |
| polyoxyethylene | | | | | | | | | |
| Polymer 3: | — | — | 3.5 | — | — | 3.5 | — | — | — |
| Polyethylene glycol- | | | | | | | | | |
| 100 | | | | | | | | | |
| Stearyl ether dimer | | | | | | | | | |
| Isophorone | | | | | | | | | |
| diisocyanate | | | | | | | | | |
| Polymer 4: | — | — | — | 3 | — | — | — | — | — |
| Polyethylene glycol- | | | | | | | | | |
| 150/Stearyl | | | | | | | | | |
| alcohol/Methylene | | | | | | | | | |
| diphenyl | | | | | | | | | |
| diisocyanate | | | | | | | | | |
| Polymer 5: | — | — | — | — | 5 | — | — | — | — |
| Polyethylene glycol- | | | | | | | | | |
| 150/Decyl | | | | | | | | | |
| alcohol/Methylene | | | | | | | | | |
| diphenyl | | | | | | | | | |
| diisocyanate | | | | | | | | | |
| Polymer 6: | — | — | — | — | — | — | 15 | — | — |
| Hydroxypropylcellulose | | | | | | | | | |
| Polymer 7: | — | — | — | — | — | — | — | — | — |
| Acrylate/C10-C30 | | | | | | | | | |
| alkyl acrylate | | | | | | | | | |
| Polymer 8: | — | — | — | — | — | — | — | — | — |
| carboxyvinylic | | | | | | | | | |
| Polymer 9: | — | — | — | — | — | — | — | — | — |
| Polyacrylate-3 | | | | | | | | | |
| Aminomethyl | — | — | — | — | — | — | — | — | — |
| propanol | | | | | | | | | |
| Ethanol | 4.3 | 4.3 | 3.0 | 3.0 | 3.0 | 5.0 | Q.S. 100 | 2.6 | 2.6 |
| Distilled water | Q.s. 100 | Q.s. 100 | Q.s. 100 | Q.s. 100 | Q.s. 100 | Q.s. 100 | 20.4 | Q.s. 100 | Q.s. 100 |
| Viscosity (cP) | 4338 | 14768 | 7370 | 7840 | 8141 | 8210 | 8371 | 3140 | 8540 |
| Turbidity (NTU) | 880 | 198 | 282 | 302 | 784 | 223 | 202 | 414 | 158 |
| Appearance of gel | 2 | 1 | 1 | 1 | 2 | 1 | 1 | 1 | 1 |

| | | | | | |
|---|---|---|---|---|---|
| 27 | 28 | 29 | 30 | 31 | 32 |

| | |
|---|---|
| Compositions | COMPARATIVE |

| | | | | | | |
|---|---|---|---|---|---|---|
| A2 | 10 | 10 | 7 | 7 | 6 | 6 |
| Polymer 1: | — | — | — | — | — | — |
| Hydroxypropyl guar | | | | | | |
| Polymer 2: | — | — | — | — | — | — |
| Hexamethyl | | | | | | |
| diisocyanate/polyethylene | | | | | | |
| glycol with | | | | | | |
| alpha-omega | | | | | | |
| termination | | | | | | |
| Stearyl | | | | | | |
| polyoxyethylene | | | | | | |
| Polymer 3: | — | — | — | — | — | — |
| Polyethylene glycol- | | | | | | |
| 100 | | | | | | |
| Stearyl ether dimer | | | | | | |
| Isophorone | | | | | | |
| diisocyanate | | | | | | |
| Polymer 4: | — | — | — | — | — | — |
| Polyethylene glycol- | | | | | | |
| 150/Stearyl | | | | | | |
| alcohol/Methylene | | | | | | |
| diphenyl | | | | | | |
| diisocyanate | | | | | | |
| Polymer 5: | — | — | — | — | — | — |
| Polyethylene glycol- | | | | | | |
| 150/Decyl | | | | | | |
| alcohol/Methylene | | | | | | |
| diphenyl | | | | | | |
| diisocyanate | | | | | | |
| Polymer 6: | — | — | — | — | — | — |
| Hydroxypropylcellulose | | | | | | |
| Polymer 7: | 1 | — | 1 | — | 1.6 | — |
| Acrylate/C10-C30 | | | | | | |
| alkyl acrylate | | | | | | |
| Polymer 8: | — | — | — | — | — | 0.6 |
| carboxyvinylic | | | | | | |
| Polymer 9: | — | 0.7 | — | 0.7 | — | — |
| Polyacrylate-3 | | | | | | |

| | |
|---|---|
| Aminomethyl | QS pH7 |
| propanol | |

| | | | | | | |
|---|---|---|---|---|---|---|
| Ethanol | 4.3 | 4.3 | 3.0 | 3.0 | 2.6 | 2.6 |
| Distilled water | Q.s. 100 | Q.s. 100 | Q.s. 100 | Q.s. 100 | Q.s. 100 | Q.s. 100 |
| Viscosity (cP) | 2322 | 10354 | 1523 | 8582 | 2597 | 5677 |

| | |
|---|---|
| Turbidity (NTU) | >1000 (not measurable, limit of |
| | detection of the apparatus) |

| | | | | | | |
|---|---|---|---|---|---|---|
| Appearance of gel | 3 | 3 | 3 | 3 | 3 | 3 |

| | 33 | 34 | 35 | 36 |
|---|---|---|---|---|
| Composition | INVENTION | | | COMPARATIVE |
| A3 | 7 | 7 | 7 | 7 |
| Polymer 2: Hexamethyl | 3.5 | | | |
| diisocyanate/polyethylene glycol with | | | | |
| alpha-omega termination/stearyl | | | | |
| polyoxyethylene | | | | |
| Polymer 6: | | 15 | | |
| Hydroxypropylcellulose | | | | |
| Polymer 3: | | | 3.5 | |
| Polyethylene glycol-100 | | | | |
| Stearyl ether dimer | | | | |
| Isophorone diisocyanate | | | | |
| Polymer 9: | | | | 2.5 |
| Polyacrylate-3 | | | | |

| | |
|---|---|
| Aminomethyl propanol | QS pH7 |

| Ethanol | — | Q.s. 100 | 2 | — |
|---|---|---|---|---|
| Distilled water | Q.s. 100 | — | Q.s. 100 | Q.s. 100 |
| Viscosity (cP) | 10372 | 4008 | 3610 | 3380 |
| Turbidity (NTU) | 281 | 185 | 259 | >1000 (not |
| | | | | measurable, limit of |
| | | | | detection of the |
| | | | | apparatus) |
| Appearance of gel | 1 | 1 | 1 | 3 |
| | 37 | 38 | 39 | 40 |
| Composition | INVENTION | | | COMPARATIVE |
| A4 | 7 | 7 | 7 | 7 |
| Polymer 2: Hexamethyl | 3.5 | — | — | — |
| diisocyanate/polyethylene glycol with | | | | |
| alpha-omega termination/stearyl | | | | |
| polyoxyethylene | | | | |
| Polymer 6: | — | 15 | — | — |
| Hydroxypropylcellulose | | | | |
| Polymer 3: | — | — | 3.5 | — |
| Polyethylene glycol-100 | | | | |
| Stearyl ether dimer | | | | |
| Isophorone diisocyanate | | | | |
| Polymer 8: | — | — | — | 1.5 |
| Carboxyvinylic | | | | |
| Aminomethyl propanol | — | — | — | QS pH7 |
| Ethanol | — | Q.s. 100 | 2 | — |
| Distilled water | Q.s. 100 | — | Q.s. 100 | Q.s. 100 |
| Viscosity (cP) | 6593 | 3610 | 2670 | White gel of creamy |
| | | | | texture, and |
| | | | | inhomogeneous |
| | | | | (formation of lumps) |
| Turbidity (NTU) | 132 | 56 | 128 | >1000 (not |
| | | | | measurable, limit of |
| | | | | detection of the |
| | | | | apparatus) |
| Appearance of gel | 1 | 1 | 1 | 3 |

| | | | |
|---|---|---|---|
| 41 | 42 | 43 | 44 |

| | | |
|---|---|---|
| Composition | INVENTION | COMPARATIVE |

| | | | | |
|---|---|---|---|---|
| A5 | 7 | — | 7 | — |
| A6 | — | 7 | — | 7 |
| Polymer 2: Hexamethyl | — | — | — | — |
| diisocyanate/polyethylene glycol | | | | |
| with alpha-omega | | | | |
| termination/stearyl | | | | |
| polyoxyethylene | | | | |
| Polymer 6: | 15 | 15 | — | — |
| Hydroxypropylcellulose | | | | |
| Polymer 8: | — | — | 3 | 2.5 |
| Carboxyvinylic | | | | |
| Ethanol | Q.s. 100 | Q.s. 100 | — | — |
| Distilled water | — | — | Q.s. 100 | Q.s. 100 |
| Viscosity (cP) | 9480 | 9104 | 5537 | 5490 |

| | | | |
|---|---|---|---|
| Turbidity (NTU) | 216 | 46 | >1000 (not measurable, limit of |
| | | | detection of the apparatus) |
| Appearance of gel | 1 | 1 | 3 |

### Protocol for Application of the Compositions at Room Temperature

Locks of hair of natural chestnut colour were used; 2.7 g; not coloured, not bleached and not permed.

1. Carried out standard shampooing.

2. Rinsed the hair in water.

3. Dried the hair with a towel.

4. Applied the composition on the moist lock of hair with a bath ratio of 0.5.

5. Left the hair to dry in the open air or else with a hood or a hair-dryer.

### Results

For each of the compositions according to the present disclosure containing an elastomeric anionic film-forming polyurethane (A) and a non-ionic thickening polymer, the gel obtained was homogeneous and sufficiently thick to permit quick and easy application of the composition on the hair. Moreover, the product flowed well in the hands and on the hair, so as to obtain a uniform distribution on all of the hair.

During application and after drying, there was significant shaping of the hair and maintenance of this shaping.

The hair fixing was elastic and flexible. The polymer film formed was non-brittle and therefore had high fixing power, which was long-lasting.

What is claimed is:

1. A cosmetic composition comprising, in a cosmetically acceptable medium, at least one elastomeric anionic film-forming polyurethane (A) and at least one non-ionic thickening polymer, wherein the at least one elastomeric anionic film-forming polyurethane (A) comprises:
(i) at least one side chain comprising at least one ethylene oxide unit, wherein the at least one side chain
represents from 12 to 80% by weight of polyurethane (A) and
has a Mw ranging from 1000 g/mol to 30,000 g/mol; and
(ii) one main chain comprising units derived from:
a polypropylene glycol (PPG) and optionally a second non-ionic polyol;
a dihydroxy-carboxylic acid; and
at least one di-isocyanate; and optionally
a chain extender organic polyamine having an average of at least 2 primary amine groups.

2. The cosmetic composition according to claim 1, wherein the film obtained by drying the at least one elastomeric anionic film-forming polyurethane (A) at room temperature (24° C.±2° C.) and at a relative humidity of 48%±5%, has a mechanical profile comprising:
(a) an elongation at break (ε_{b}) of greater than or equal to 730%±5%;
(b) an instantaneous recovery (Rᵢ) of greater than or equal to 70%±5%, after an elongation of 150%; and
(c) a recovery (R₃₀₀) at 300 seconds of greater than 80%±5%; and
wherein the at least one elastomeric anionic film-forming polyurethane is soluble in water or is soluble in a mixture of water and ethanol comprising less than 30% by weight of ethanol.

3. The cosmetic composition according to claim 2, wherein the at least one elastomeric anionic film-forming polyurethane (A) is present in an amount ranging from 0.05 to 20 wt. % relative to the total weight of the cosmetic composition

4. The composition according to claim 3, wherein the at least one elastomeric anionic film-forming polyurethane (A) is present in an amount ranging from 1 to 10 wt. % relative to the total weight of the cosmetic composition.

5. The cosmetic composition according to claim 2, wherein the at least one non-ionic thickening polymer is chosen from associative polymers.

6. The cosmetic composition according to claim 5, wherein the at least one non-ionic thickening polymer is chosen from:
(1) celluloses modified with groups comprising at least one fatty chain;
(2) non-ionic guar gums comprising at least one fatty chain;
(3) copolymers of vinyl pyrrolidone and hydrophobic fatty-chain monomers;
(4) copolymers of C₁-C₆ alkyl methacrylates or acrylates and amphiphilic monomers comprising at least one fatty chain;
(5) copolymers of hydrophilic methacrylates or acrylates and hydrophobic monomers comprising at least one fatty chain;
(6) polyether polyurethanes comprising both hydrophilic sequences and hydrophobic sequences in their chain; and
(7) polymers with an aminoplast ether backbone comprising at least one fatty chain.

7. The cosmetic composition according to claim 2, wherein the the at least one non-ionic thickening polymer is present in an amount ranging from 0.01 to 5 wt. % relative to the total weight of the composition.

8. The cosmetic composition according to claim 7, wherein the at least one non-ionic thickening polymer is present in an amount ranging from 0.1 to 2 wt. %, relative to the total weight of the composition.

9. The cosmetic composition according to claim 2, wherein the pH of the cosmetic composition ranges from 6 to 9.

10. The cosmetic composition according to claim 2, wherein the cosmetically acceptable medium comprises water and/or at least one organic solvent chosen from C₂ to C₄ alcohols, polyols, polyol ethers, acetone, propylene carbonate, and benzyl alcohol.

11. The cosmetic composition according to claim 2, further comprising at least one additive chosen from ionic surfactants; non-ionic surfactants; cationic thickeners; anionic thickeners; amphoteric thickeners; penetrating agents; perfumes; colorants; plasticizers; buffers; waxes; volatile or non-volatile, cyclic or linear or branched, organically modified or unmodified silicones; ceramides; pseudoceramides; vegetable oils; mineral oils; synthetic oils; vitamins; provitamins; opacifiers; reducing agents; emulsifiers; preservatives; mineral fillers; nacres; glitter; sun filters; proteins; anionic, non-ionic, cationic or amphoteric fixing polymers different from the anionic polyurethanes (A); hydrating agents; emollients; softening agents; antifoaming agents; antiperspirants; anti-free-radical agents; bactericides; sequestering agents; anti-dandruff agents; antioxidants; alkalizing agents; and acidifying agents.

12. The cosmetic composition according to claim 2, wherein the cosmetic composition displays a turbidity at 25° C. ranging from 0 and 900 NTU units.

13. The cosmetic composition according to claim 2, wherein the cosmetic composition displays a turbidity at 25° C. ranging from 0 and 500 NTU units.

14. The cosmetic composition according to claim 2, wherein the cosmetic composition is a non-rinse composition.

15. A process for shaping keratin fibers comprising: applying to the keratin fibers a cosmetic composition, in a cosmetically acceptable medium, comprising:
at least one elastomeric anionic film-forming polyurethane (A) and at least one non-ionic thickening polymer, wherein:
the at least one elastomeric anionic film-forming polyurethane (A) comprises:
(i) at least one side chain comprising at least one ethylene oxide unit, wherein the at least one side chain
represents from 12 to 80% by weight of polyurethane (A) and
has a Mw ranging from 1000 g/mol to 30,000 g/mol; and
(ii) one main chain comprising units derived from:
a polypropylene glycol (PPG) and optionally a second non-ionic polyol;
a dihydroxy-carboxylic acid; and
at least one di-isocyanate; and optionally
a chain extender organic polyamine having an average of at least 2 primary amine groups; and
further wherein the film obtained by drying the at least one elastomeric anionic film-forming polyurethane (A) at room temperature (24° C.±2° C.) and at a relative humidity of 48%±5%, has a mechanical profile comprising:
(a) an elongation at break (ε_{b}) of greater than or equal to 730%±5%;
(b) an instantaneous recovery (Ri) of greater than or equal to 70%±5%, after an elongation of 150%; and
(c) a recovery (R₃₀₀) at 300 seconds of greater than 80%±5%; and
wherein the at least one elastomeric anionic film-forming polyurethane is soluble in water or is soluble in a mixture of water and ethanol comprising less than 30% by weight of ethanol.
